(19) United States
(12) Patent Application Publication
(10) Pub. No.: US 2008/0227713 A1
(43) Pub. Date: Sep. 18, 2008

(54) Oxidized Human Bnp

(76) Inventor: Andrew A. Protter, Palo Alto, CA (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 12/089,192

(22) PCT Filed: Oct. 3, 2006
(86) PCT No.: PCT/US06/38860

§ 371 Date: Apr. 3, 2008

Related U.S. Application Data
(60) Provisional application No. 60/723,094, filed on Oct. 3, 2005.

(51) Int. Cl. A61K 38/16; C07K 14/00; C07K 16/00; G01N 33/68; G01N 33/53; C12P 21/00; A61P 9/00

(52) U.S. Cl. 514/12; 530/324; 530/387.9; 436/86; 436/501; 435/69.1

(57) ABSTRACT

The invention disclosed herein relates to oxidized forms of brain natriuretic peptide (BNP), particularly human BNP (hBNP). The disclosed invention relates to oxidized forms of B-type natriuretic peptide (BNP), which is useful as a marker for cardiovascular disease

### TECHNICAL FIELD

The disclosed invention relates to oxidized forms of B-type natriuretic peptide (BNP), which is useful as a marker for cardiovascular disease.

### BACKGROUND ART

Natriuretic peptides comprise a family of vasoactive hormones which play important roles in the regulation of cardiovascular and renal homeostasis. Atrial natriuretic peptide (ANP) and B-type natriuretic peptide (BNP) are predominantly produced in the heart and exert vasorelaxant, natriuretic, and antigrowth activities. It has been demonstrated that plasma BNP levels correlate with the severity of heart failure. However, the precise structure of circulating BNP-immunoreactive material has not been determined.

Human BNP (hBNP) consists of a 32 amino acid peptide with a 17 amino acid disulfide loop structure. The peptide contains two methionine residues, one at position 4 and the second at position 15. Human BNP is initially translated in the cell as a 134 amino acid protein containing a 26 amino acid signal peptide which presumably is rapidly removed during synthesis (Seilhamer et. al., Biochem Biophys Res Commun 165:650-658 (1989); Sudoh et al., Biochem Biophys Res Commun 159:1427-1434 (1989)). Once the signal peptide is removed a 108 amino acid BNP precursor protein, termed proBNP, is produced with the 32 amino acid BNP peptide located at the carboxyl-terminal end. It is generally believed that the heart secretes a mixture of the proBNP protein as well as the mature BNP peptide into the blood.

Plasma immuno-reactive BNP levels have been shown to correlate with the severity of heart failure (Mukoyama et al., N Engl J Med 323:757-758 (1990) and Cowie et al., Eur Heart J. (19):1710-8 (2003) for review). However the molecular nature of this material is unclear. Hino et al., (BBRC 167:693-700 (1990)) purified immuno-reactive BNP from human cardiac atria and by amino acid sequence analysis characterized the unprocessed 108 amino acid proBNP protein as well as the 32 amino acid peptide, suggesting the heart releases both the mature peptide hormone and the precursor protein (proBNP) into the blood. Several studies have shown that a high molecular weight form of BNP, probably the 108 amino acid proBNP, circulates in the blood of both healthy individuals and heart failure patients (Yandle et al. J Clin Endocrinol Metab 76:832-838 (1993); Togashi et al. Clin Chem Acta. 37:765 (1991); Togashi et al. Clin Chem 39:550-551 (1993); Shimizu et al. Clin Chem Acta 316:129-135 (2002)). The molecular nature of the low molecular weight BNP-immunoreactive material remains unclear. Analyzing BNP-immunoreactive material in plasma from 3 individuals with heart failure failed to demonstrate the presence of any of the mature 32 amino acid form (Shimizu et al. Clin Chem Acta 316:129-135 (2002)).

BNP exerts it biological effects by activating a specific cell surface receptor termed the guanylyl cyclase-A (GC-A) receptor or the NPR-A receptor. When activated, the receptor synthesizes cyclic GMP from GTP. Treatment of cells with BNP increases intracellular and extracellular concentrations of cyclic GMP. Furthermore, treatment of animals with BNP results in dose-dependent increases in cyclic GMP in the plasma. It is generally believed that the GC-A receptor and cyclic GMP mediates most if not all of the biological effects of BNP.

### SUMMARY OF THE INVENTION

The invention disclosed herein relates to oxidized forms of brain natriuretic peptide (BNP), particularly human BNP (hBNP). One embodiment of the disclosed invention relates to an isolated and purified peptide having natriuretic activity, comprising the formula: Ser¹-Pro²-Lys³-Met⁴-Val⁵-Gln⁶-Gly⁷-Ser⁸-Gly⁹-Cys¹⁰-Phe¹¹-Gly¹²-Arg¹³-Lys¹⁴-Met¹⁵-Asp¹⁶-Arg¹⁷-Ile¹⁸-Ser¹⁹-Ser²⁰-Ser²¹-Ser²²-Gly²³-Leu²⁴-Gly²⁵-Cys²⁶-Lys²⁷-Val²⁸-Leu²⁹-Arg³⁰-Lys³¹-His³², wherein either Met⁴, Met¹⁵, or both are oxidized. One aspect of this embodiment includes pharmaceutical compositions for inducing natriuresis, diuresis and/or vasodilation in a subject which composition comprises an effective amount of the disclosed oxidized hBNP peptide in admixture with a suitable pharmaceutical excipient.

Another embodiment of the disclosed invention relates to detection reagents and their use for detecting heart failure. One aspect of this embodiment encompasses an isolated antibody or fragment thereof that is monospecifically reactive to hBNP, wherein the antibody binds specifically to a peptide of the formula: Ser¹-Pro²-Lys³-Met⁴-Val⁵-Gln⁶-Gly⁷-Ser⁸-Gly⁹-Cys¹⁰-Phe¹¹-Gly¹²-Arg¹³-Lys¹⁴-Met¹⁵-Asp¹⁶-Arg¹⁷-Ile¹⁸-Ser¹⁹-Ser²⁰-Ser²¹-Ser²²-Gly²³-Leu²⁴-Gly²⁵-Cys²⁶-Lys²⁷-Val²⁸-Leu²⁹-Arg³⁰-Lys³¹-His³², wherein either Met⁴, Met¹⁵, or both are oxidized. In a particular aspect, the antibody or fragment thereof of claims 3 or 4, wherein the antibody is monospecifically reactive with Met¹⁵-Asp¹⁶-Arg¹⁷-Ile¹⁸-Ser¹⁹-Ser²⁰-Ser²¹-Ser²²-Gly²³-Leu²⁴-Gly²⁵. In another aspect of this embodiment, the antibody or fragment thereof of claims 3 or 4, wherein the antibody is monospecifically reactive with Val⁵-Gln⁶-Gly⁷-Ser⁸-Gly⁹-Cys¹⁰-Phe¹¹-Gly¹²-Arg¹³. A variety of antibodies are contemplated by the presently disclosed invention. Examples of such antibodies include polyclonal antibodies, monoclonal antibody, and functional antibody fragments such as Fab, F(ab′)₂ and Fab′, Fv or sFv fragment.

Another embodiment of the disclosed invention relates to the detection of oxidized forms of hBNP as an indicator of cardiovascular disease, such as heart failure. One aspect of this embodiment relates to a method to diagnosis heart failure, comprising detecting the presence or absence of an oxidized form of hBNP in a sample, wherein the presence of oxidized hBNP is an indicator of heart failure. The method encompassed detecting the oxidized forms of hBNP, wherein Met⁴, Met¹⁵, or both are oxidized. In one embodiment, an immunoassay is used to detect the presence of oxidized forms of hBNP.

Another embodiment of the disclosed invention relates to a method to produce an oxidized hBNP peptide having natriuretic activity, comprising providing a recombinant host cell which has been manipulated to contain an expression system of which expresses a peptide of the formula: Ser¹-Pro²-Lys³-Met⁴-Val⁵-Gln⁶-Gly⁷-Ser⁸-Gly⁹-Cys¹⁰-Phe¹¹-Gly¹²-Arg¹³-Lys¹⁴-Met¹⁵-Asp¹⁶-Arg¹⁷-Ile¹⁸-Ser¹⁹-Ser²⁰-Ser²¹-Ser²²-Gly²³-Leu²⁴-Gly²⁵-Cys²⁶-Lys²⁷-Val²⁸-Leu²⁹-Arg³⁰-Lys³¹-His³², wherein either Met⁴, Met¹⁵, or both are oxidized; culturing the cells under conditions which permit the expression of the peptide; recovering the peptide from the culture; and oxidizing the peptide, such that Met⁴, Met¹⁵ or both are oxidized.

Still another embodiment of the disclosed invention relates to a method of treating a subject for a condition characterized by an abnormally high extracellular fluid level which method comprises administering to said subject an effective amount of a pharmaceutical composition comprising a peptide of the formula: Ser¹-Pro²-Lys³-Met⁴-Val⁵-Gln⁶-Gly⁷-Ser⁸-Gly⁹-Cys¹⁰-Phe¹¹-Gly¹²-Arg¹³-Lys¹⁴-Met¹⁵-Asp¹⁶-Arg¹⁷-Ile¹⁸-Ser¹⁹-Ser²⁰-Ser²¹-Ser¹²-Gly²³-Leu²⁴-Gly²⁵-Cys²⁶-Lys²⁷-Val²⁵-Leu²⁹-Arg³⁰-Lys³¹-His³², wherein either Met⁴, Met¹⁵, or both are oxidized, whereby extracellular fluid levels decrease.

A further embodiment relates to a method of producing elevated levels of oxidize hBNP in plasma, comprising administering to a subject an effective amount of pharmaceutical composition comprising a peptide of the formula: Ser¹-Pro 2-Lys³-Met⁴-Val⁵-Gln⁶-Gly⁷-Ser⁸-Gly⁹-Cys¹⁰-Phe¹¹-Gly¹²-Arg¹³-Lys¹⁴-Met¹⁵-Asp¹⁶-Arg¹⁷-Ile¹⁸-Ser¹⁹-Ser²⁰-Ser²¹-Ser²²-Gly²³-Leu²⁴-Gly²⁵-Cys²⁶-Lys²⁷-Val²⁸-Leu²⁹-Arg³⁰-Lys³-His³², wherein either Met⁴, Met¹⁵, or both are oxidized, whereby elevated plasma levels of the oxidized hBNP results.

### BRIEF DESCRIPTION OF THE DRAWINGS

FIG. 1. LCMS of the Heart Failure patient plasma sample. Total ion current (TIC) of the heart failure patient plasma sample is shown. The peak that contains BNP and oxidized forms of BNP is indicated with arrow.

FIG. 2. Ion extraction of BNP with quadruplet charges from Heart Failure patient plasma sample. Extracted ion current (XIC) of the heart failure patient plasma sample is shown. The peak that contains BNP and oxidized forms of BNP is indicated with arrow.

FIG. 3. BNP and Oxidized BNP spectrum from Heart Failure patient plasma sample. Averaged spectrum under BNP and oxidized BNP peaks from heart failure patient plasma sample. The ion signal that corresponding to BNP is indicated with arrow.

FIG. 4. Zoomed-in spectrum for BNP and its oxidized BNP signal from Heart Failure patient plasma sample. This is the zoomed-in spectrum for the ions of BNP and oxidized BNP with quadruplet charges contained in FIG. 3. The spectrum clearly showed three clusters of ions at the m/z 867, 870 and 875 region that corresponding to quadruplet charged BNP, oxidized-BNP and di-oxidized-BNP, respectively. Different ions in each ion cluster represent the isotopic distribution of each molecule.

FIG. 5 (A or B). Deconvoluted spectrum of BNP and Oxidized BNP from Heart Failure patient plasma sample. The spectrum in FIG. 3 were deconvoluted to obtain molecular weight of the ions detected. Three major cluster of masses were obtained that were consistent with the expected molecular weight of BNP, oxidize-BNP and di-oxidized-BNP, respectively.

FIG. 6. Deconvoluted mass list of BNP and Oxidized BNP spectrum from Heart Failure patient plasma sample. The masses of the spectrum shown in FIG. 3 is listed in the table after deconvolution calculation. The table showed that the three major masses were derived from both isotope ion distribution and multiply charged ion series (IC).

FIG. 7. LCMS of the normal human plasma spiked with BNP standard sample. Total ion current (TIC) of the normal human plasma sample spiked with BNP reference standard is shown. The peak that contains BNP is indicated with arrow.

FIG. 8. Ion extraction of BNP with quadruplet charges from normal human plasma spiked with BNP standard. Extracted ion current (XIC) of the normal human plasma spiked with BNP standard sample is shown. The peak that contains BNP is indicated with arrow.

FIG. 9. BNP spectrum from normal human plasma spiked with BNP standard sample. Averaged spectrum under BNP peak from normal human plasma spiked with BNP standard sample. The ion signal that corresponding to BNP is indicated with arrow.

FIG. 10. Zoomed-in spectrum for BNP signal from normal human plasma spiked with BNP standard sample. This is the zoomed-in spectrum for the ions of BNP with quadruplet charges contained in FIG. 9. Unlike that shown in FIG. 4, only one ion clusters of ions at the m/z 867 region that corresponding to quadruplet charged BNP can be observed.

FIG. 11(A or B). Deconvoluted spectrum of BNP from normal human plasma spiked with BNP standard sample. The spectrum in FIG. 9 were deconvoluted to obtain molecular weight of the ions detected. Only one major cluster of masses were obtained that were consistent with the expected molecular weight of BNP and its isotopic isomers.

FIG. 12. Deconvoluted mass list of BNP spectrum from normal human plasma spiked with BNP standard sample. The masses of the spectrum shown in FIG. 9 is listed in the table after deconvolution calculation. The table showed that only one major mass were derived from the spectrum).

FIG. 13. Stimulation of the Human GC-A Receptor with hBNP and [Met(O)⁴]-hBNP.

FIG. 14. Stimulation of the Human GC-A Receptor with hBNP and [Met(O)¹⁵]-hBNP.

FIG. 15. Pharmacokinetics of [Met(O)¹⁵]-hBNP vs. hBNP in Rabbits

FIG. 16. Steady State Values as a Function of Infusion Rate for BNP and [Met(O)¹⁵]-hBNP.

FIG. 17. Plasma Cyclic GMP During Continuous Infusion of [Met(O)¹⁵]-hBNP and hBNP.

FIG. 18. Plasma Cyclic GMP Levels Resulting from a Single Intravenous Bolus 30-μg/kg Dose of hBNP, [Met(O)¹⁵]-hBNP, or [Met(O)⁴]-hBNP Administered to Conscious Rabbits.

FIG. 19. Amino Acid Sequence Comparison of Human ANP (28 Amino Acid Form) and Human BNP.

### DESCRIPTION OF THE PREFERRED EMBODIMENTS

The disclosed invention relates to oxidized forms of human B-type natriuretic peptide (BNP) and uses for such peptides. Analogs of human BNP in which either of the two methionine residues at positions 4 and 15 are replaced by a methionine-sulphoxide moiety, [Met(O)⁴]-hBNP and [Met(O)¹⁵]-hBNP, are formed during the NATRECOR® hBNP manufacturing process. The structural similarity of these two impurities to human BNP suggested that they might have biological activity. As disclosed herein, oxidized forms of hBNP have utility as markers for heart disease and heart failure.

It is shown here that oxidized forms of hBNP exist naturally in the blood and that oxidized forms of hBNP have utility as diagnostic markers of cardiovascular disease. Heart failure is an example of a cardiovascular disease in which levels of oxidized BNP increase. Heart failure is a progressive disorder in which damage to the heart causes weakening of the cardiovascular system. It is clinically manifested by fluid congestion or inadequate blood flow to tissues.

The 32 amino acid human BNP peptide contains two methionine residues at positions 4 and 15. Oxidation of either or both of these methionine residues produces one or more methionine-sulphoxide residues. Oxidation of either or both of these methionine residues produces three forms of BNP: oxidation of the methionine at position 4 ([Met-O⁴]hBNP), oxidation of the methionine at position 15 ([Met-O¹⁵]hBNP) and oxidation at both methionine ([Met-O^{4,15}]hBNP).

The heart produces hBNP with one or more of the native methionine residues being oxidized or reduced. In subjects experiencing cardiovascular disease, such as heart failure, the subjects' hearts release oxidized forms of hBNP into the blood at higher than normal levels. As such, detection of these oxidized forms of BNP serves as an indication of cardiovascular disease.

Detection of the oxidized forms of BNP in the blood of a subject can be achieved by a variety of methods that are well known in the art. Typically, a subject who is experiencing or is thought to be experiencing a cardiovascular disease state provides a sample for analysis. A blood sample is most preferred, but any bodily fluid that contains BNP can be used in an assay for oxidized forms of hBNP.

A preferred method of detecting the present of oxidized forms of hBNP utilizes an immunochemical methodology. ELISA assays, RIAs, and other well known immunologically based assays can be used to identify the present of an oxidized form of hBNP in a sample. Column chromatographic techniques that are or are not based on immunological principles can also be used to determine whether oxidized forms of hBNP are present in a sample.

Antibodies Against Oxidized hBNP

This invention provides highly sensitive reagents which allow for the rapid, simple and accurate quantification of oxidized forms hBNP at clinically relevant titers in biological fluids such as plasma or serum. Preferably, antibodies which recognize an oxidized epitope presented by hBNP are used as the reagents. Any epitope comprising a methionine residue which may be oxidized can be used to generate the reagents. In particular, previously identified highly immunogenic epitopes within the hBNP molecule can be used to produce antibodies monospecific to the relevant. When an antibody is referred to herein as being “monospecific” to an epitope, it is meant that the antibody is capable of binding a sequence of amino acids within oxidized hBNP containing the amino acids comprising the epitope, but is incapable of binding to a sequence of amino acids within hBNP that does not contain the amino acids comprising the epitope. The epitopes recognized by the monospecific antibodies of the invention typically comprise amino acids 5-13 and 15-25 of hBNP, which contain the MET residues as position 4 and 15, respectively. However, it should be recognized that any antigenic peptide derived from hBNP that contains the relevant residues can be used as an antigen for generating antibodies that recognize the oxidized forms of hBNP. The monospecific antibodies of the invention are preferably, though not necessarily, monoclonal antibodies.

In a preferred embodiment, an antibody is generated against hBNP and that antibody is used to screen the subject's sample for the presence of an oxidized form of hBNP. The term “antibody” is used in the broadest sense. Therefore an “antibody” can be naturally occurring or man-made such as monoclonal antibodies produced by conventional hybridoma technology. Anti-hBNP antibodies comprise monoclonal and polyclonal antibodies as well as fragments containing the antigen-binding domain and/or one or more complementarity determining regions of these antibodies. An “antibody fragment” is defined as at least a portion of the variable region of the immunoglobulin molecule that binds to its target, i.e., the antigen-binding region. A particularly preferred embodiment of the present invention encompasses antibodies that are immunospecific for [Met-O⁴]hBNP or [Met-O¹⁵]hBNP.

Various methods for the preparation of antibodies are well known in the art. For example, antibodies can be prepared by immunizing a suitable mammalian host using a hBNP peptide or fragment, in isolated or conjugated form (Antibodies: A Laboratory Manual, CSH Press, Eds., Harlow, and Lane (1988); Harlow, Antibodies, Cold Spring Harbor Press, NY (1989)). In addition, fusion proteins of hBNP can also be used, such as a hBNP::GST-fusion protein. In addition, naked DNA immunization techniques known in the art are used (with or without purified hBNP or hBNP expressing cells) to generate an immune response to the encoded immunogen. Examples of antibodies against hBNP are discussed in U.S. Pat. Nos. 6,124,430 and 6,162,902, both of which are hereby incorporated by reference in its entirety.

Monoclonal antibodies of the invention can be produced by hybridoma cells prepared according to known procedures, e.g. Kohler, G. and Milstein, C., Nature, 256:495, 1975. In general, mice are immunized with an immunogenic conjugate of hBNP and a suitable partner, such as bovine serum albumin. Periodic booster injections are administered until good antibody titers are achieved. Spleen cells from the immunized mice are then fused with myeloma cells according to known procedures (i.e. Galfre et al., Nature, 266:550, 1977) to produce hybridoma cells. Supernatants from the hybridoma cell cultures are screened for reactivity to hBNP. Hybridomas testing positive are recloned and their supernatants retested for reactivity. Using this procedure, as described in more detail below, we obtained hybridoma cell line 106.3, which secretes monoclonal antibodies that specifically recognize the hBNP fragment 5-13, hybridoma cell line 201.3, which secretes monoclonal antibodies that specifically recognize the hBNP fragment 1-10 and hybridoma cell line 8.1, which secretes monoclonal antibodies that specifically recognize the hBNP fragment 27-32.

Once a highly immunogenic epitope has been identified, non-monoclonal monospecific antibodies can be produced from polyclonal antisera. Polyclonal antisera is produced according to known techniques. A suitable animal, such as a rabbit, is immunized with an immunogenic conjugate of hBNP and a suitable partner. Periodic booster injections are administered until good antibody titers are achieved. By testing the antisera thus obtained for immunoreactivity against various peptide fragments of hBNP, desirable epitopes are identified. Using this procedure, we identified the peptide fragment hBNP 15-25 as a highly immunogenic epitope of hBNP. Monospecific antibodies to the identified epitope can be obtained by affinity purification of polyclonal serum on an affinity column in which a peptide fragment including only that epitope and none of the other epitopes identified in the epitope mapping is bound to a solid support. Using this general procedure, as described in more detail below, we produced non-monoclonal monospecific antibodies that specifically recognize the hBNP fragment 15-25.

It will be recognized, however, that monoclonal antibodies to this epitope can also be produced using known procedures.

Functionally active fragments of the monospecific antibodies of the invention can also be used in assays for hBNP. A functional fragment is one that retains the immunologic specificity of the antibody, although avidity and/or affinity may not be quantitatively identical. Included in the functionally active fragments are such immunoglobulin fragments as Fab, F(ab′)₂ and Fab′. The fragments can be produced by known methods such as by enzymatic cleavage of the monospecific antibodies (see, e.g., Mariani, M. et al., Mol. Immunol., 28:69-77 (1991); Ishikawa, E. et al., J. Immunoassay, 4:209-327 (1983)).

### Immunoassays

The detection reagents of the invention can be used to carry out immunoassays to quantify oxidized hBNP levels in biological fluids such as plasma, serum and whole blood. The assays can be carried out using a variety of art recognized techniques, such as a sandwich type format or in a competition format. When measuring oxidized hBNP levels in blood, the biological fluid is preferably plasma or serum, which can be prepared from whole blood using known procedures.

In a sandwich type assay, two different antibodies are employed to separate and quantify the oxidized hBNP in the biological fluid sample. In one embodiment, the two antibodies bind to the oxidized hBNP, thereby forming an immune complex, or sandwich. Generally, one of the antibodies is used to capture the oxidized hBNP in the sample and a second antibody is used to bind a quantifiable label to the sandwich. Preferably, the antibodies chosen to carry out the sandwich type assay are selected such that the first antibody which is brought into contact with the oxidized hBNP-containing sample does not bind all or part of the epitope recognized by the second antibody, thereby significantly interfering with the ability of the second antibody to bind oxidized hBNP. Consequently, if a sandwich type format is chosen, one preferably should not employ an antibody monospecific for hBNP 5-13 in combination with an antibody monospecific for hBNP 1-10 inasmuch as the epitopes recognized by these antibodies overlap. It has been found, however, that an excellent assay can be effected using a monospecific antibody as the first antibody brought into contact with the oxidized hBNP-containing sample and a high affinity polyclonal antibody for hBNP1-32 as the second antibody. In particular, a sandwich type assay which is sensitive in the range of clinically relevant hBNP titers using a monoclonal antibody which recognizes the epitope hBNP 5-13 as a capture antibody and a rabbit polyclonal antibody to hBNP as the second antibody has been developed. This assay can readily be converted to detecting oxidized forms of hBNP using the appropriate antibodies rather than antibodies which recognize any form of hBNP.

Production and Preparation of Oxidized hBNP

Production of hBNP is achieved using techniques that are well known in the art. For example, the hBNP production techniques discussed in U.S. Pat. No. 5,674,710, which is hereby incorporated by reference in its entirety, can be used to produce hBNP. Alternatively, well known synthetic methods of producing peptides can be used to prepare hBNP are available. Similarly, preparation of oxidized forms of hBNP, containing one or more oxidized methionine residues can be achieved using standardized chemical reactions that are well known in the art.

A purified preparation of oxidized hBNP is contemplated as an embodiment of the presently disclosed invention. Preferably, a preparation of hBNP that contains more than approximately 9 μg/mg of [Met-O⁴]hBNP and/or more than approximately 7 μg/mg of [Met-O¹⁵]hBNP comprises a purified preparation of oxidized hBNP. In other terms, preparations containing from 1% to 100% oxidized forms of either [Met-O⁴]hBNP or [Met-O¹⁵]hBNP are contemplated as comprising purified preparations of oxidized hBNP. Accordingly, hBNP preparations comprising 5%, 10%, 15%, 20%, 25%, 30%, 35%, 40%, 45%, 50%, 55%, 60%, 65%, 70%, 75%, 80%, 85%, 90%, 95% or more of one or more forms of oxidized hBNP are contemplated as a purified preparation of oxidized BNP.

Biological Activity of Oxidized hBNP

Human BNP is known to affect plasma cyclic GMP, reducing blood pressure, diuresis, and natriuresis. As discussed below, it is clear that oxidized forms of hBNP demonstrate altered biochemical effects, including increased plasma stability.

Previous studies have demonstrated that hBNP induces a dose-related release of cyclic GMP from cells expressing the human guanylyl cyclase-A (GC-A), consistent with reports demonstrating that the GC-A receptor mediates most and probably all of the biological effects of hBNP and that cyclic GMP is an important second messenger for this receptor. In this study, the effects of hBNP, [Met(O)⁴]-hBNP, and [Met(O)¹⁵]-hBNP on cyclic GMP release from cells expressing the human GC-A receptor were determined.

Previous studies using rabbits as an animal model have described pharmacokinetics and biological responses to hBNP including stimulation of plasma cyclic GMP, reducing blood pressure, diuresis, and natriuresis. Thus, in this study, the pharmacokinetics and biological effects of hBNP, [Met(O)¹⁵]-hBNP, and [Met(O)⁴]-hBNP were determined and compared.

The hBNP variants [Met(O)⁴]-hBNP and [Met(O)¹⁵]-hBNP are present in the hBNP drug product. These impurities might by pharmacologically active as they are structurally similar to hBNP. This report describes the in vitro and in vivo pharmacology of [Met(O)⁴] hBNP and [Met(O)¹⁵]-hBNP. In vitro, using cells expressing the human GC-A receptor, [Met(O)⁴]-hBNP was shown to be equivalent to hBNP in inducing cellular cyclic GMP release, a measure of receptor activation. [Met(O)¹⁵]-hBNP was far less potent than hBNP in this assay, indicating that it is a much poorer ligand for the biological receptor of hBNP. These results are consistent with the animal data showing that with regard to induction of plasma cyclic GMP, a measure of GC-A receptor activation in vivo, [Met(O)⁴]-hBNP and hBNP were comparable while [Met(O)¹⁵]-hBNP was significantly less effective.

It appears that pharmacokinetics does not explain the weak induction of plasma cyclic GMP by [Met(O)¹⁵]-hBNP. In rabbits, [Met(O)¹⁵]-hBNP appeared to be more stable in the plasma compartment than hBNP as evidenced by the higher steady state plasma levels achieved at equivalent doses of these two peptides. The increased values for the estimated steady state of [Met(O)¹⁵]-hBNP compared to hBNP in rabbits suggest that [Met(O)¹⁵]-hBNP may achieve a higher plasma level in humans as well. As the estimated steady state values for [Met(O)¹⁵]-hBNP were between 1.6- and 1.9-fold greater than the values derived for hBNP using identical infusion rates, the predicted change in clearance rate in humans compared to hBNP is relatively small. There were no remarkable differences between the pharmacokinetics of [Met(O)⁴]-hBNP and hBNP.

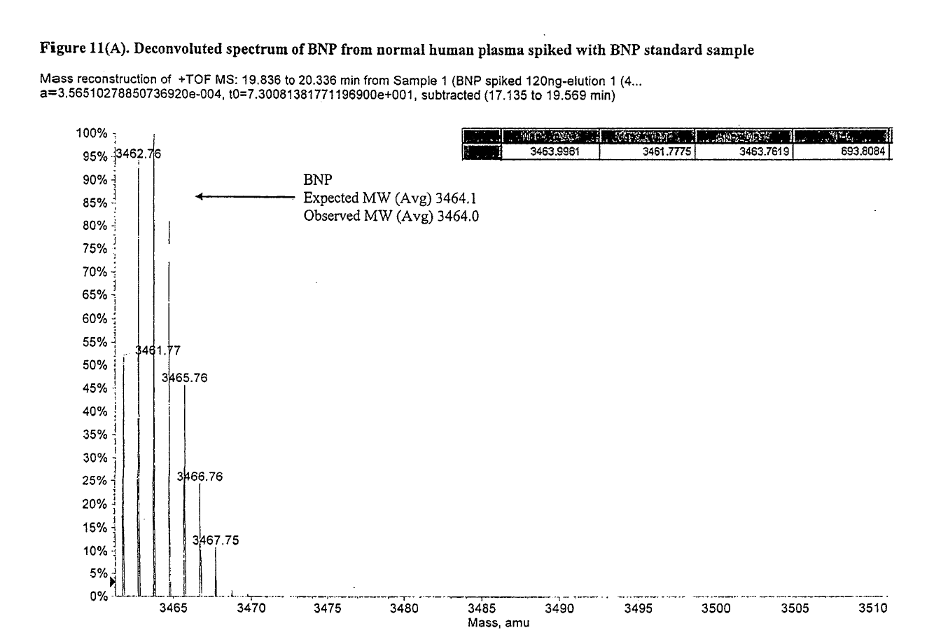

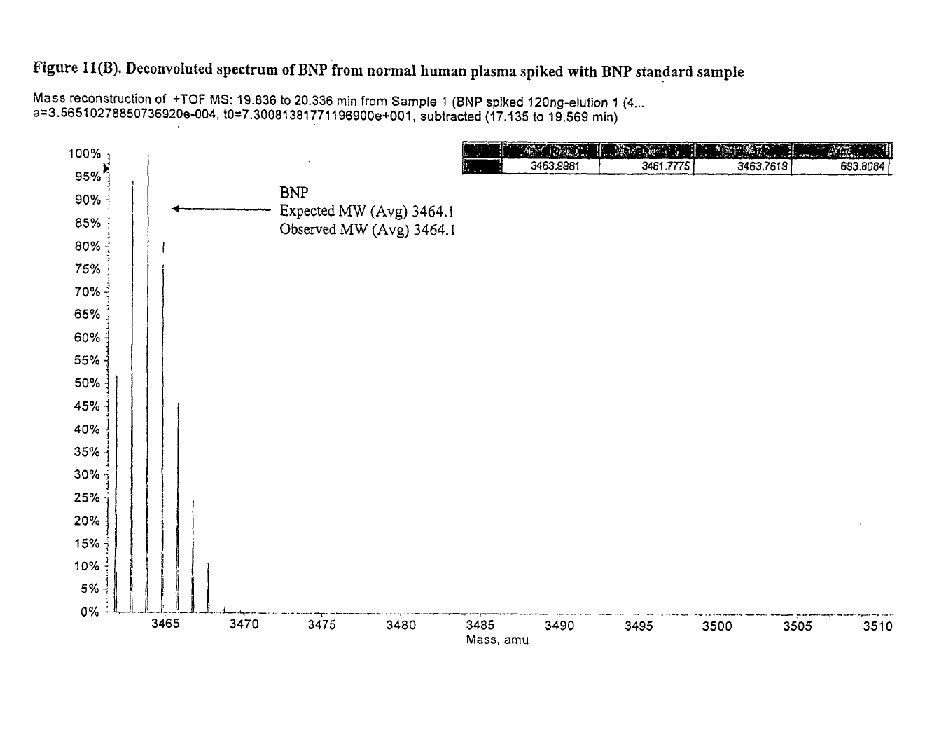

Previous in vitro and in vivo assays have shown that a single oxidation of the methionine residue at position 12 in human ANP, termed [Met-O¹²]ANP, significantly reduces the activity of the peptide (Biochem Biophys Res Commun, 128:538-46 (1985); Mol Pharmacol, 47:172-80 (1995); Eur J Biochem, 203:425-32 (1992); J Clin Invest, 83:482-9 (1989); and Eur J Pharmacol, 147:49-57 (1988)). ANP and BNP are structurally similar, with the methionine at position 15 in human BNP corresponding to the methionine at position 12 in human ANP (FIG. 11). This region of the ANP peptide is believed to be important for activation of the (GC-A) receptor, the biological receptor for both ANP and BNP. Numerous reports have shown that [Met-O¹²]ANP has a reduced ability to stimulate cyclic GMP production in cultured cells expressing rat, murine, or human GC-A receptors. Using cells expressing the human GC-A receptor, researchers reported that [Met-O¹²]ANP had only 10% the potency of human ANP in stimulating cyclic GMP.

Consistent with these cell culture studies are reports that [Met-O¹²]ANP has reduced activity in relaxing pre-contracted vascular tissue preparations when compared to human ANP. (Eur J Pharmacol, 147:49-57 (1988)) In studies in intact rats, [Met-O¹²]ANP has significantly reduced diuretic and natriuretic actions. It is interesting to note that in the one study in which blood pressure was measured, [Met-O¹²]ANP and human ANP had similar hypotensive actions. (J Clin Invest, 83:482-9 (1989)) The low potency of [Met(O)¹⁵]-hBNP with regard to cyclic GMP induction and renal effects is consistent with what has been reported for [Met-O¹²]ANP. The reason for the normal hemodynamic effects of [Met-O¹²]ANP and [Met(O)¹⁵]-hBNP is unclear.

In summary, [Met(O)¹⁵]-hBNP has significantly reduced actions against the biological receptor of hBNP. When compared to hBNP, [Met(O)¹⁵]-hBNP has reduced actions in rabbits with regard to induction of plasma cyclic GMP, natriuresis, and diuresis.

### Administration

Briefly, the natriuretic peptides of the invention are useful in treatment of disorders associated with high levels of extracellular fluids such as hypertension. The peptides disclosed herein can be used for the intravenous treatment of patients with acutely decompensated congestive heart failure who have dyspnea at rest or with minimal activity. In this population, the use of the disclosed peptides will reduce pulmonary capillary wedge pressure and improve dyspnea. The compounds are administered in conventional formulations for peptides such as those described in Remington's Pharmaceutical Sciences, Mack Publishing Company, Easton, Pa. (latest edition). Preferably, the peptides are administered by injection, preferably intravenously, using appropriate formulations for this route of administration. Dosage levels are on the order of 0.01-100 μg/kg of subject.

These compounds, and compositions containing them, can find use as therapeutic agents in the treatment of various edematous states such as, for example, congestive heart failure, nephrotic syndrome and hepatic cirrhosis, in addition to hypertension and renal failure due to ineffective renal perfusion or reduced glomerular filtration rate. The natriuretic peptides of the invention are particularly effective in the treatment of congestive heart failure.

Thus the present invention also provides compositions containing an effective amount of compounds of the present invention, including the nontoxic addition salts, amides and esters thereof, which may, alone, serve to provide the above-recited therapeutic benefits. Such compositions can also be provided together with physiologically tolerable liquid, gel or solid diluents, adjuvants and excipients.

These compounds and compositions can be administered to mammals for veterinary use, such as with domestic animals, and clinical use in humans in a manner similar to other therapeutic agents. In general, the dosage required for therapeutic efficacy will range from about 0.001 to 100 μg/kg, more usually 0.01 to 100 μg/kg of the host body weight. Alternatively, dosages within these ranges can be administered by constant infusion over an extended period of time, usually exceeding 24 hours, until the desired therapeutic benefits have been obtained.

Typically, such compositions are prepared as injectables, either as liquid solutions or suspensions; solid forms suitable for solution in, or suspension in, liquid prior to injection may also be prepared. The preparation may also be emulsified. The active ingredient is often mixed with diluents or excipients which are physiologically tolerable and compatible with the active ingredient. Suitable diluents and excipients are, for example, water, saline, dextrose, glycerol, or the like, and combinations thereof. In addition, if desired the compositions may contain minor amounts of auxiliary substances such as wetting or emulsifying agents, stabilizing or pH-buffering agents, and the like.

The compositions are conventionally administered parenterally, by injection, for example, either subcutaneously or intravenously. Additional formulations which are suitable for other modes of administration include suppositories, intranasal aerosols, and, in some cases, oral formulations. For suppositories, traditional binders and excipients may include, for example, polyalkylene glycols or triglycerides; such suppositories may be formed from mixtures containing the active ingredient in the range of 0.5% to 10% preferably 1%-2%. Oral formulations include such normally employed excipients as, for example, pharmaceutical grades of mannitol, lactose, starch, magnesium stearate, sodium saccharin, cellulose, magnesium carbonate, and the like. These compositions take the form of solutions, suspensions, tablets, pills, capsules, sustained-release formulations, or powders, and contain 10%-95% of active ingredient, preferably 25%-70%.

The peptide compounds may be formulated into the compositions as neutral or salt forms. Pharmaceutically acceptable nontoxic salts include the acid addition salts (formed with the free amino groups) and which are formed with inorganic acids such as, for example, hydrochloric or phosphoric acids, or organic acids such as, for example, hydrochloric or phosphoric acids, or such organic acids as acetic, oxalic, tartaric, mandelic, and the like. Salts formed with the free carboxyl groups may be derived from inorganic bases such as, for example, sodium, potassium, ammonium, calcium, or ferric hydroxides, and such organic bases as isopropylamine, trimethylamine, 2-ethylamino ethanol, histidine, procaine, and the like.

In addition to the compounds of the present invention which display natriuretic, diuretic or vasorelaxant activity, compounds of the present invention can also be employed as intermediates in the synthesis of such useful compounds. Alternatively, by appropriate selection, compounds of the present invention whose activity levels are reduced or eliminated entirely can serve to modulate the activity of other diuretic, natriuretic or vasorelaxant compounds, including compounds outside the scope of the present invention, by, for example, binding to clearance receptors, stimulating receptor turnover, or providing alternate substrates for degradative enzyme or receptor activity and thus inhibiting these enzymes or receptors. When employed in this manner, such compounds can be delivered as admixtures with other active compounds or can be delivered separately, for example, in their own carriers.

Compounds of the present invention can also be used for preparing antisera for use in immunoassays employing labeled reagents, usually antibodies. Conveniently, the polypeptides can be conjugated to an antigenicity-conferring carrier, if necessary, by means of dialdehydes, carbodiimide or using commercially available linkers. These compounds and immunologic reagents may be labeled with a variety of labels such as chromophores, fluorophores such as, e.g., fluorescein or rhodamine, radioisotopes such as ¹²⁵I, ³⁵S, ¹⁴C, or ³H, or magnetized particles, by means well known in the art.

These labeled compounds and reagents, or labeled reagents capable of recognizing and specifically binding to them, can find use as, e.g., diagnostic reagents. Samples derived from biological specimens can be assayed for the presence or amount of substances having a common antigenic determinant with compounds of the present invention. In addition, monoclonal antibodies can be prepared by methods known in the art, which antibodies can find therapeutic use, e.g., to neutralize overproduction of immunologically related compounds in vivo.

Suitable subjects include those animals having conditions of high water or sodium ion accumulation. Both veterinary and therapeutic uses in humans are appropriate.

The following examples are offered to illustrate but not to limit the invention. All referenced cited herein are incorporated by reference in their entirety.

### Example 1

### Immunoprecipitation (IP) of Human BNP

An anti-hBNP monoclonal antibody (mab 106.3) was produced by immunizing mice with a full-length of hBNP1-32. This antibody recognizes hBNP and its fragments 1-29, 1-26, 2-32, 5-13 as well as alkylated BNP1-32 without disulfide loop. The anti-hBNP antibody was directly immobilized to an agarose gel using SEIZE PRIMARY IMMUNOPRECIPITATION KIT (PIERCE) according to the manufacture's instructions. Heart failure plasma was incubated with the hBNP antibody coupled gel overnight at 4° C., and centrifuged at 5000×g. The pellet was washed three times with immunoprecipitation buffer (IP buffer) containing 0.025 M Tris and 0.15 M NaCl at pH 7.2, and eluted with PIERCE elution buffer. The eluted samples from six patients were pooled and filtered using 30 kD MICROCON CENTRIFUGAL FILTER SYSTEM (MILLIPORE) to remove large proteins such as albumin and IgG. For the control, standard hBNP was spiked into normal human plasma and immunoprecipitated and eluted as described above. BNP concentration in the eluted samples was measured using hBNP EIA kit (PHOENIX PHARMACEUTICALS). Twelve nanograms of eluted immunoreactive (ir) hBNP from heart failure patient's plasma and 14 ng of eluted standard BNP spiked in normal human plasma were submitted to mass spectrum analysis.

After purification, the pH of the IP purified samples was adjusted with 10% formic acid to pH 3. The sample was loaded onto a VYDAC C18 capillary column (0.5×50 mm) through multiple injections. The sample was then eluted with a gradient of water with 0.2% formic acid and 0.01% TFA (Solvent A) and acetonitrile with 0.2% formic acid and 0.01% TFA (solvent B) on AGILENT 1100 capillary HPLC instrument. Specifically, the sample was loaded under 1% solvent B with 10 μl/min flow rate. After the sample loading, the flow rate was changed to 5 μl/min through out the solvent gradient program. The solvent B increased to 5% in 0.5 minute after the starting of the gradient program and kept at 5% solvent B for 5 minutes. The solvent B was then increased to 60% in the following 35 minutes to elute the sample from the column. The Solvent B was decreased to 1% at 40.1 minutes and the column was equilibrated with 1% solvent B for 20 minutes.

### Example 2

### Mass Spectrometric (MS) Analysis

Human BNP(1-32) and oxidized forms of human BNP(1-32) were immuno-precipitate purified from plasma sample from heart failure patients using BNP specific monoclonal antibody, as described in Example 1. The liquid chromatography (LC) eluent from Example 1 was split through an on-line splitter with 1:9 split ratio. About 0.5 ul/min of the HPLC eluent was fed onto a QSTAR mass spectrometer equipped with electrospray ion source. As a positive control, normal human plasma spiked with BNP(1-32) reference standard was prepared according to the method of Example 1. The BNP spiked sample was purified and liquid chromatographic mass spectrometry (LCMS) analyzed in a similar manner as that for the heart failure patient sample. The mass spectrometer was set at time-of flight (TOF) mode with the mass scan range m/z 500 to 1800. The spray voltage was set at 4 Kv and the ion accumulation time was set at 2 seconds.

The MS data showed the presence of BNP(1-32) with the molecular weight of 3464.1 amu, oxidized BNP as well as di-oxidized BNP were also observed with molecular weights of 3480.1 amu and 3496.0 amu, respectively. Only BNP(1-32) with a molecular weight of 3464.1 amu was detected in the control sample. This data indicates that the mono-oxidized and di-oxidized forms of BNP were not generated from the IP purification procedure or during the LCMS analysis. Thus, the oxidized form of BNP(1-32) is present in the circulating blood of patients experiencing heart failure.

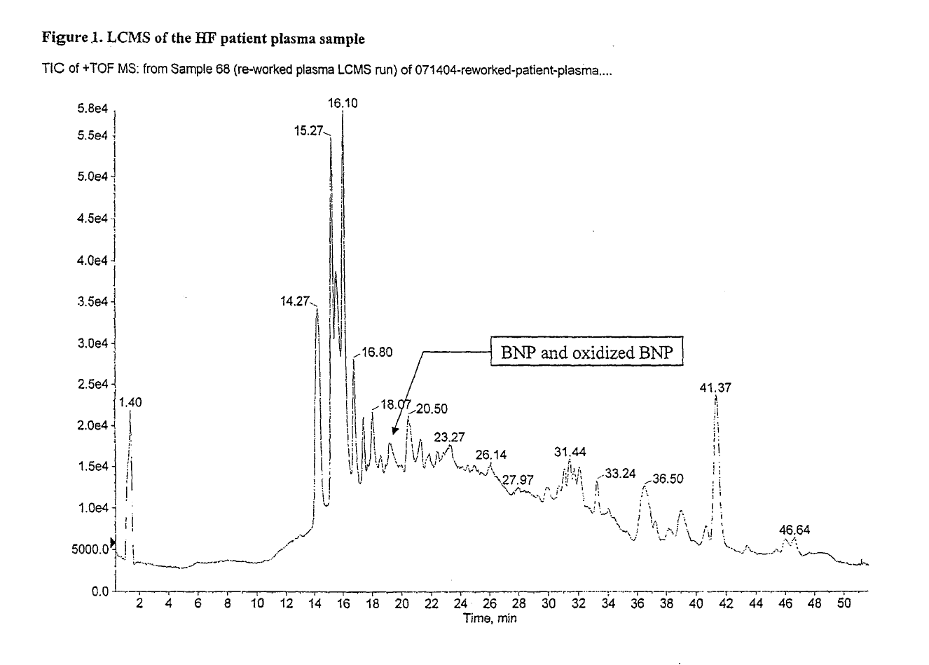

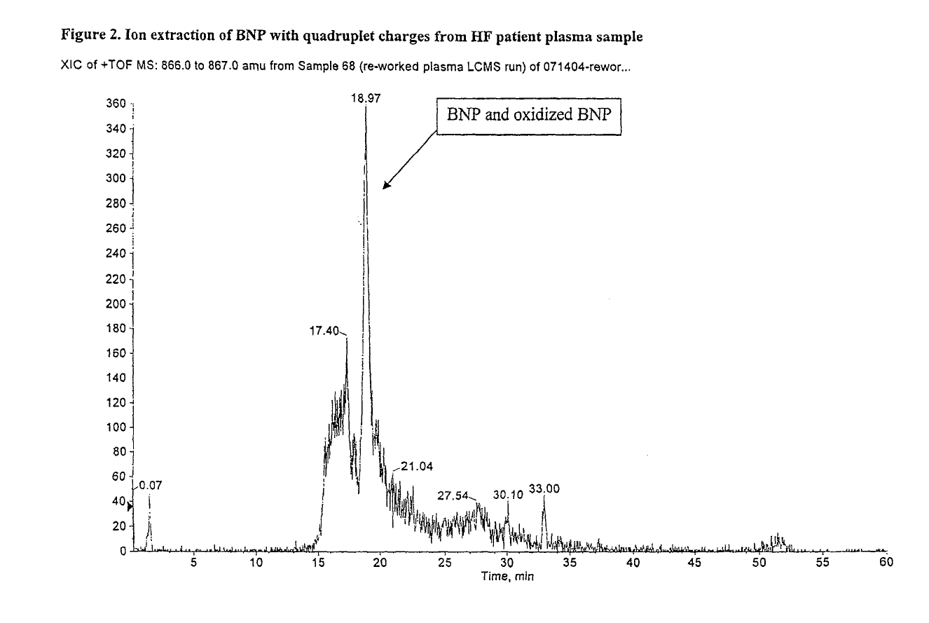

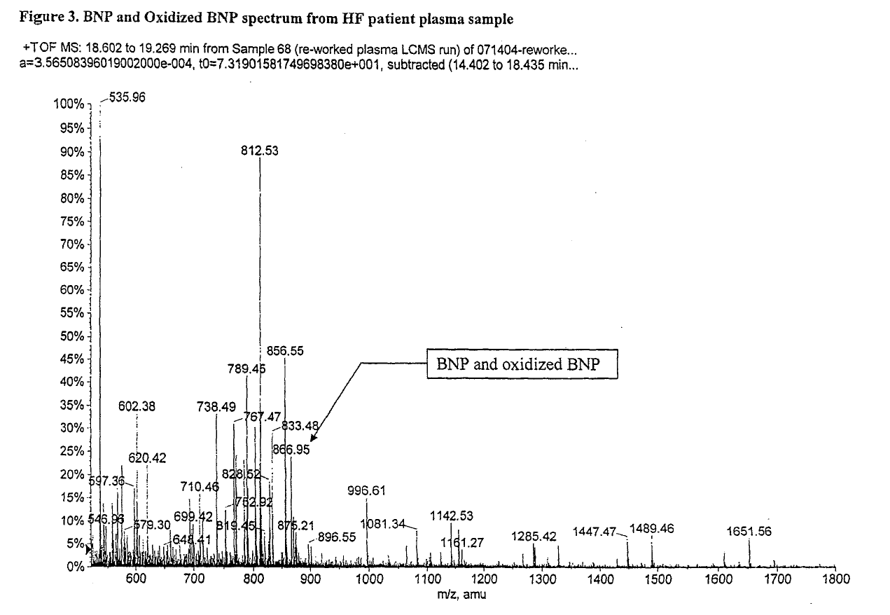

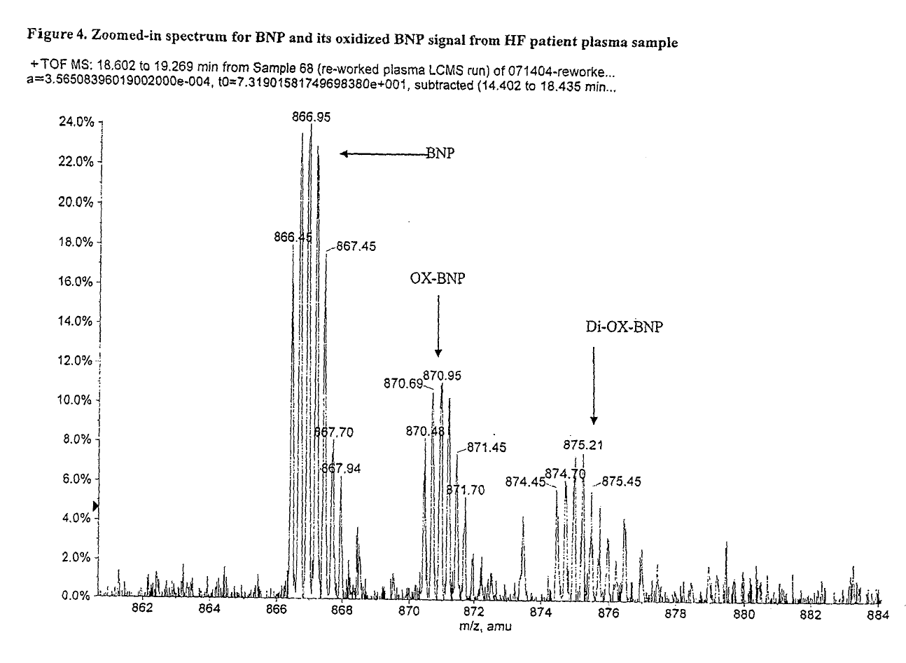

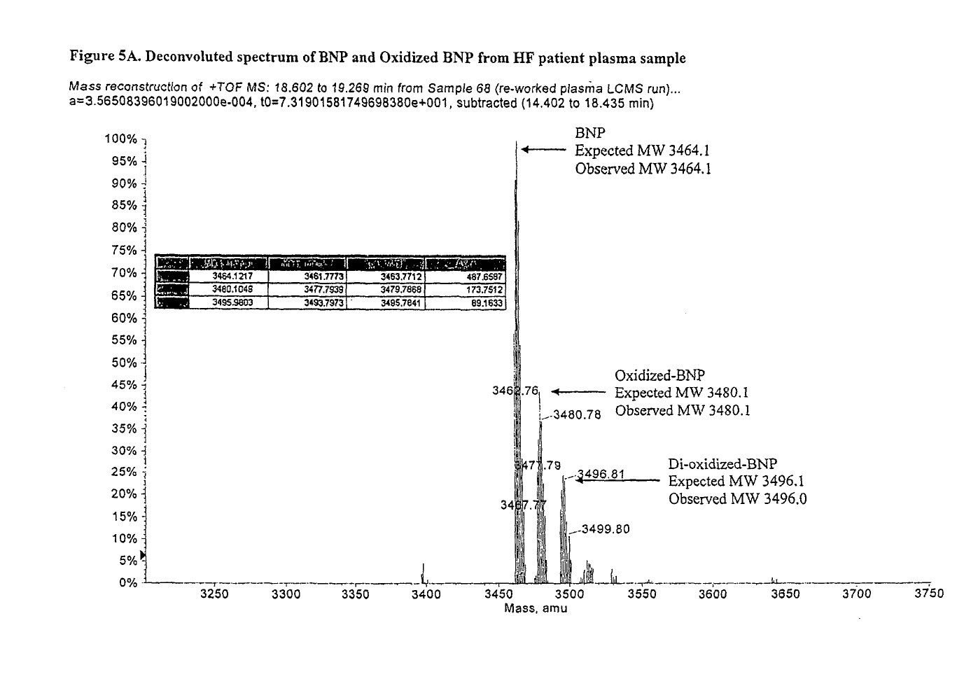

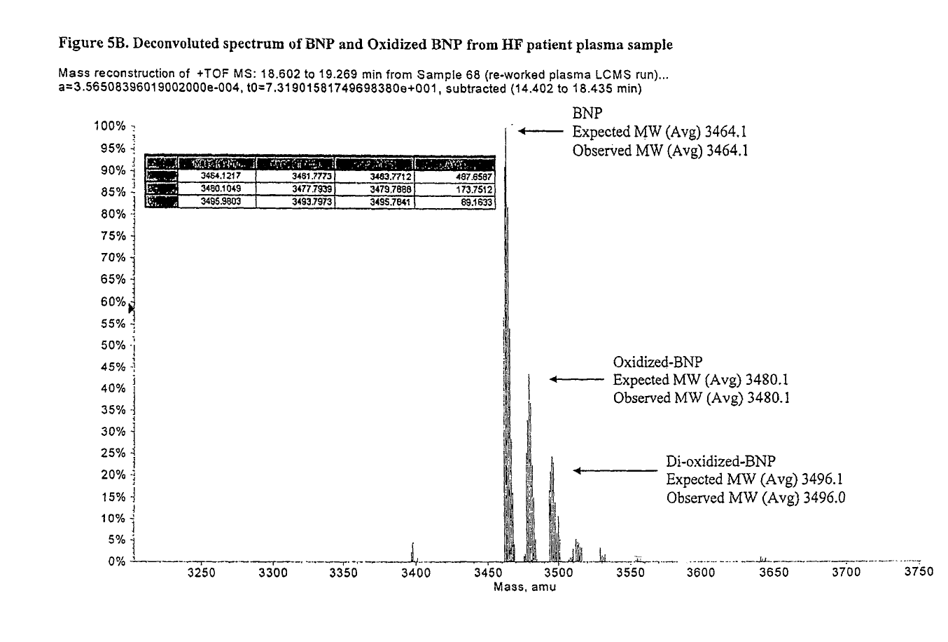

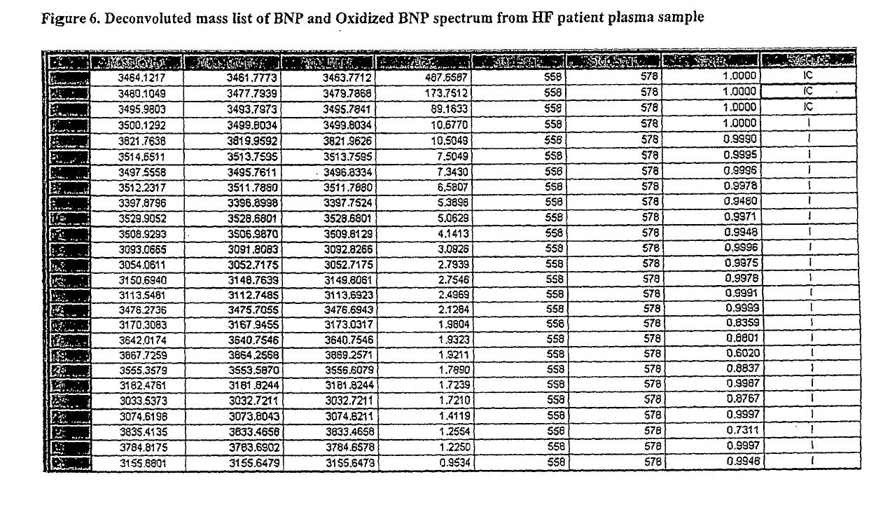

The total ion current (TIC) LCMS profile of the heart failure patient plasma sample is showed in FIG. 1. Using ion extraction method, the MS signal of m/z (mass to charge ratio) in a range of 866.5 to 866.9, corresponding to quadruplet charged ion of BNP(1-32) molecule, was obtained from TIC profile and shown in FIG. 2. The averaged MS spectrum of the extracted peak at 18.9 minutes is displayed in FIG. 3. The BNP(1-32) ions with 4 and 5 protons can be observed in the spectrum with m/z around 866.9 and 693.5, respectively. FIG. 4 showed the zoomed-in spectrum of FIG. 3 for the quadruplet charged ions of BNP (m/z 866.9) and oxidized BNP. The spectrum clearly showed three clusters of ions at the m/z 867, 870 and 875 region that corresponding to quadruplet charged BNP, oxidized-BNP and di-oxidized-BNP, respectively. Different ions in each ion cluster represent the isotopic distribution of each type of the molecule. Similar ion cluster pattern in m/z 693.5 region, that corresponding to the BNP molecule with 5 protons, can also be observed in spectrum in FIG. 3 (data not shown). Using isotopic ion cluster and charged ion series spectrum shown in FIG. 3, molecular weight of each MS signal were calculated (deconvoluted). The distribution of the masses were graphed in FIG. 5 (A or B) and tabulated in FIG. 6, which clearly shows 3 major masses that were consistent with the expected molecular weight of BNP, oxidized-BNP and di-oxidized-BNP.

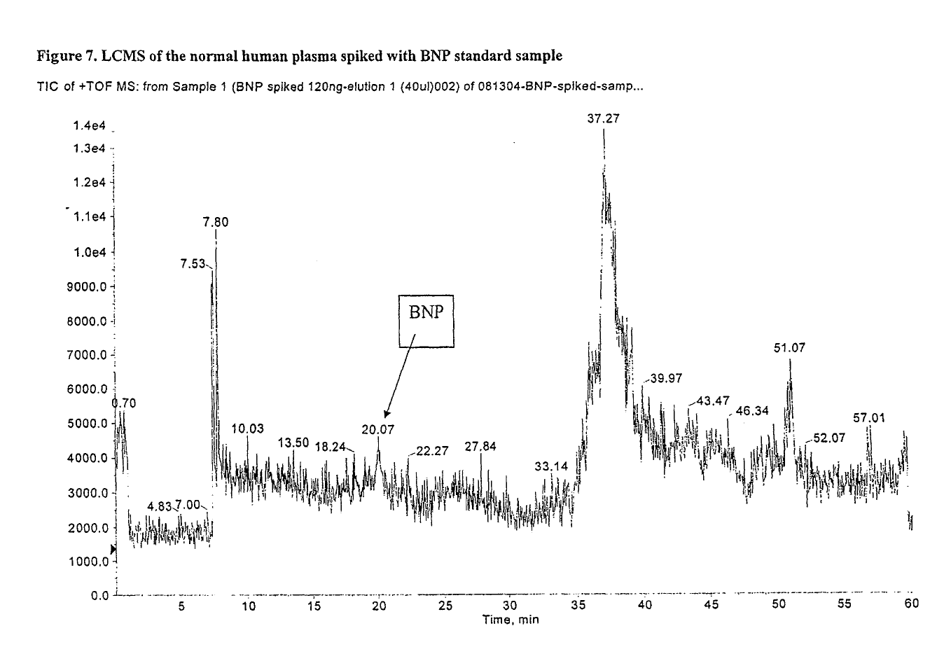

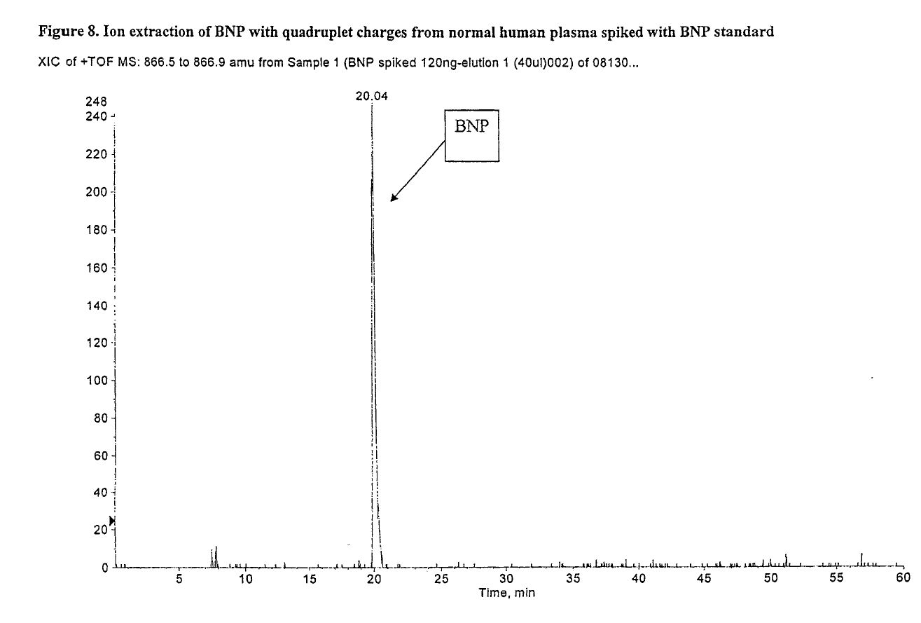

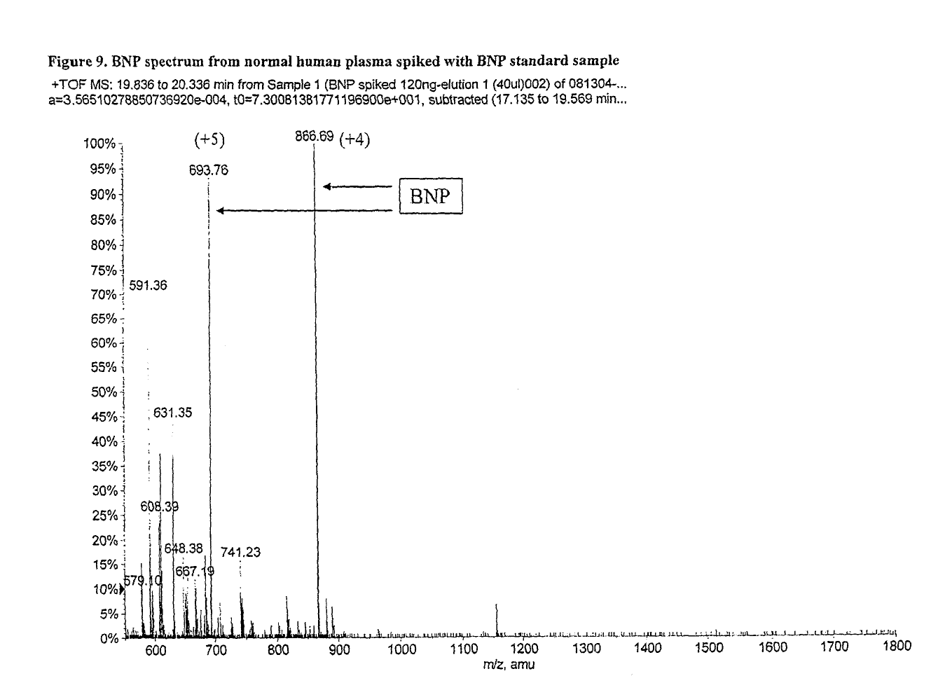

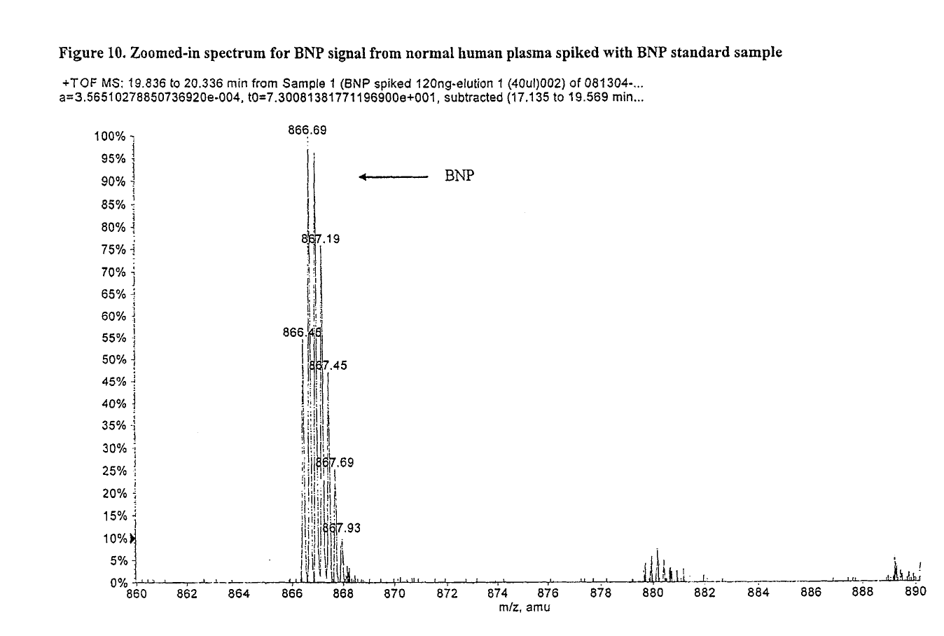

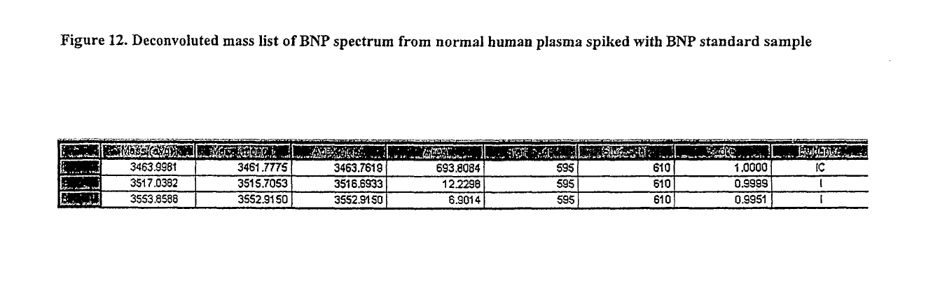

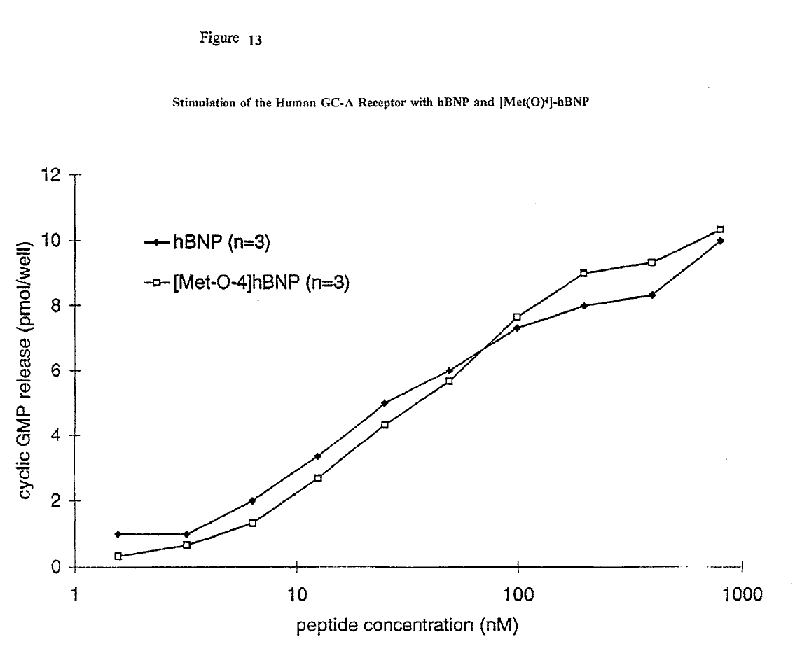

The MS data of the BNP reference standard spiked normal human plasma sample were analyzed in the same way and shown in FIG. 7 to FIG. 12. As it shown clearly in FIG. 10, only one isotopic ion cluster was observed that corresponding to quadruplet charged BNP(1-32). As expected, only one major mass was obtained after mass deconvolution under BNP peak (FIG. 11A or B) that is consistent with MW of BNP(1-32).

Mass spectrum results from plasma containing the spiked un-oxidized standard BNP showed that there is only one major cluster of masses (average MW: 3464.0) consistent with the expected molecular weight of hBNP (3464.1) in the eluted sample from normal human plasma spiked with standard hBNP, suggesting the purification procedure did not generate hBNP oxidation. In the purified samples from heart failure patient's plasma, three major cluster of masses (average MW 3464.1, MW 3480.1, and MW 3496.0) were observed. These masses are consistent with the expected molecular weights of unoxidized hBNP (MW3464.1), oxidized hBNP (3480.1), and di-oxidized hBNP (3496.1), respectively. Based on mass area, approximate 35% of hBNP in heart failure patient's plasma was oxidized (23%) or di-oxidized (12%). These data indicate that the oxidized forms of hBNP, indeed, existed in heart failure patient's plasma.

### Example 3

### Preparation of Oxidized Forms of Human BNP: [Met(O)⁴]-hBNP and [Met(O)¹⁵]-hBNP

The peptides [Met(O)⁴]-hBNP and [Met(O)¹⁵]-hBNP were prepared by Star Biochemicals by synthetic peptide chemistry. The structures were confirmed by amino acid sequence analysis and mass spectrometry. No hBNP peptide was detected by mass spectrometry analysis. The purity of [Met(O)⁴]-hBNP and [Met(O)¹⁵]-hBNP was found to be 87.5% and 89%, respectively. Lot number RM6013 hBNP, with a peptide content of 0.76 mg hBNP/mg drug substance, was used for the animal studies. hBNP was Lot No. H0007A1 [Met(O)¹⁵]-hBNP and Lot No. CS173197 with a peptide content of 0.83 mg [Met(O)¹⁵]-hBNP/mg drug substance were used for the tissue culture study.

### Example 4

### Activation of the Human GC-A Receptor In Vitro

CHO GCA 5 μl cells, a Chinese hamster ovary cell line developed to express the human GC-A receptor, were used in this study. The cells were seeded in 96-well tissue culture plates with approximately 20,000 cells per well in 180 μL of serum-free growth media. The cells were incubated overnight at 37° C., in 10% CO₂. Solutions of hBNP, [Met(O)⁴]BNP, and [Met(O)¹⁵]-hBNP were prepared in phosphate buffered saline with isobutylmethylxanthine and 2% bovine serum albumin. Peptides were added to cells to achieve the following concentrations: hBNP (2, 3, 6, 12.5, 25, 50, 100, 200, 400, 800 nM), [Met(O)⁴]-hBNP (2, 3, 6, 13, 25, 50, 100, 200, 800 nM), or [Met(O)¹⁵]-hBNP (12.5, 25, 50, 100, 200, 400, 800, 1600, 3200, 6400 nM), and the cells were incubated at 37° C. After 1.5 hours, the supernatant from each well was removed and the amount of cyclic GMP was determined by immunoassay. ED₅₀ values were determined directly from a four parameter curve fit to the dose-response data.

The equation for the fit is:

y=((A−D)/(1+(X/C)^{B}))+D

with:
y=the response;
X=the dose of the test material;
A=the baseline response, for X=0;
B=the slope factor;
C=the ED₅₀, the dose producing a response halfway between A and D; and
D=the maximum response

The % bioactivity was calculated using the formula: ED₅₀ of hBNP/ED₅₀ of test sample. Statistical analysis as indicated was performed using the program INSTAT for WINDOWS version 3.0 (GraphPad Software, San Diego, Calif.).

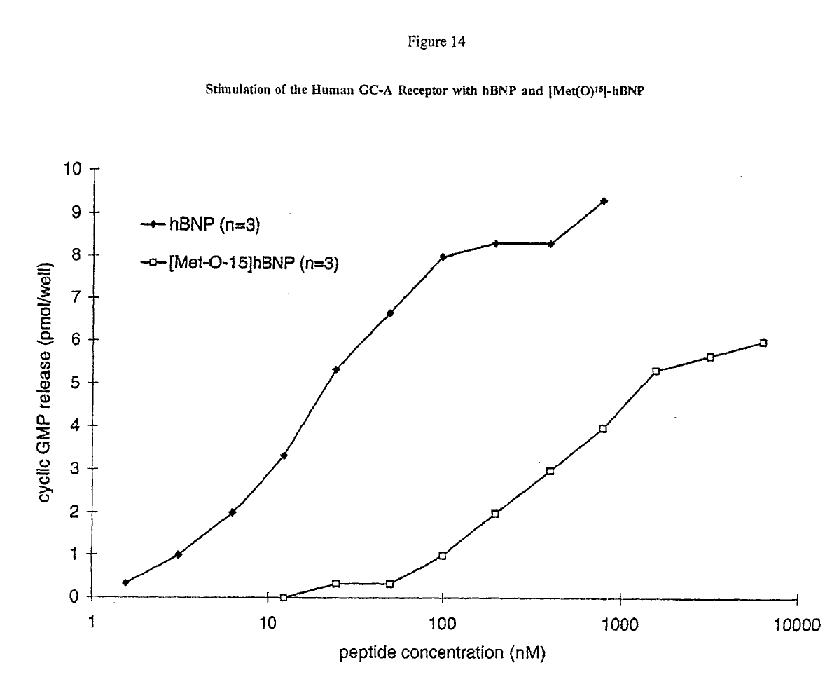

hBNP, [Met(O)⁴]-hBNP, and [Met(O)¹⁵]-hBNP induced dose-related increases in cyclic GMP accumulation in conditioned media of cells expressing the human GC-A receptor. The cyclic GMP induction profiles for [Met(O)⁴]-hBNP and hBNP were similar with regard to the magnitude of cyclic GMP induced and potency, resulting in ED₅₀ values of 39.6±10 nM and 26.2±5.6 nM, respectively (% bioactivity was 76±36) (see FIG. 1 and Tables 1 and 3). The cyclic GMP induction profile for [Met(O)¹⁵]-hBNP differed from the profile for hBNP with regard to the magnitude of cyclic GMP induced and potency, ED₅₀ values of 483±147 nM and 18.4±6.3 nM, respectively (% bioactivity was 4±1.7) (see FIG. 14 and Tables 2 and 3).

TABLE 1
STIMULATION OF THE HUMAN GC-A RECEPTOR
WITH HBNP AND [MET(O)₄]-HBNP

Cyclic GMP
(pmol/well)

| Peptide Concentration | hBNP | [Met(O)⁴]-hBNP |
|---|---|---|
| (nM) | (n = 3) | (n = 3) |
| 800 | 10.0 ± 3.5 | 10.3 ± 2.3 |
| 400 | 8.3 ± 3.2 | 9.3 ± 2.5 |
| 200 | 8.0 ± 2.6 | 9.0 ± 2.6 |
| 100 | 7.3 ± 3.2 | 7.7 ± 2.1 |
| 50 | 6.0 ± 2.0 | 5.7 ± 1.5 |
| 25 | 5.0 ± 1.0 | 4.3 ± 1.2 |
| 13 | 3.3 ± 0.6 | 2.7 ± 0.6 |
| 6 | 2.0 ± 0.0 | 1.3 ± 0.6 |
| 3 | 1.0 ± 0.0 | 0.7 ± 0.6 |
| 2 | 1.0 ± 0.0 | 0.3 ± 0.6 |

TABLE 2
STIMULATION OF THE HUMAN GC-A RECEPTOR
WITH HBNP AND [MET(O)¹⁵]-HBNP

| | |
|---|---|
| hBNP | [Met(O)¹⁵]-hBNP |

| Peptide | Cyclic GMP | Peptide | Cyclic GMP |
|---|---|---|---|
| Concentration | (pmol/well) | Concentration | (pmol/well) |
| (nM) | (n = 3) | (nM) | (n = 3) |
| 800 | 9.3 ± 3.2 | 6400 | 6.0 ± 2.0 |
| 400 | 8.3 ± 3.2 | 3200 | 5.7 ± 2.1 |
| 200 | 8.3 ± 2.5 | 1600 | 5.3 ± 1.5 |
| 100 | 8.0 ± 1.7 | 800 | 4.0 ± 1.7 |
| 50 | 6.7 ± 2.1 | 400 | 3.0 ± 1.0 |
| 25 | 5.3 ± 0.6 | 200 | 2.0 ± 1.0 |
| 13 | 3.3 ± 0.6 | 100 | 1.0 ± 0.0 |
| 6 | 2.0 ± 0.0 | 50 | 0.3 ± 0.6 |
| 3 | 1.0 ± 0.0 | 25 | 0.3 ± 0.6 |
| 2 | 0.3 ± 0.6 | 12.5 | 0.0 ± 0.0 |

TABLE 3
POTENCY AND BIOACTIVITY OF [MET(O)⁴]-HBNP
AND [MET(O)¹⁵]-HBNP RELATIVE TO HBNP
[Met(O)⁴]-hBNP

| | ED₅₀ of hBNP | | |
|---|---|---|---|
| | Reference | ED₅₀ of | |
| | Standard (nM) | [Met(O)⁴]-hBNP | % Bioactivity |
| 1 | 30.4 | 33.8 | 90 |
| 2 | 19.9 | 57.7 | 34 |
| 3 | 28.2 | 27.4 | 103 |
| Mean ± SD | 26.2 ± 5.6 | 39.6 ± 16.0 | 76 ± 36.4 |

[Met(O)¹⁵]-hBNP

| | ED₅₀ of hBNP | | |
|---|---|---|---|
| | Reference | ED₅₀ of | |
| Assay No. | Standard (nM) | [Met(O)¹⁵]-hBNP | % Bioactivity |
| 1 | 25.3 | 433 | 6 |
| 2 | 16.9 | 649 | 3 |
| 3 | 13.1 | 368 | 4 |
| Mean ± SD | 18.4 ± 6.3 | 483 ± 147 | 4 ± 1.7 |

### Example 5

### Pharmacokinetics and Pharmacodynamics of Oxidized Forms of Human BNP: [Met(O)⁴]-hBNP and [Met(O)¹⁵]-hBNP in Rabbits

New Zealand White male rabbits (R & R Rabbitry, WA) weighing 2.5 to 3.0 kg, were used in the study. Upon receipt, the animals were allowed a 1-week acclimation period at the animal facility to ensure proper health status prior to the study. On the day of the study, the rabbits were weighed and placed in an open top plexiglass restrainers with adjustable plexiglass and nylon covers. The ears were shaved, 2% Lidocaine HCl (Xylocalne jelly, Astra, Westborough, Mass.) was applied topically on the surface of the ears, and IV catheters (Angiocath, Becton Dickinson, Utah) fitted with heparin locks were inserted into the intermedial branch of the peripheral ear vein for drug administration and into the central ear artery for repeated blood sampling.

Human BNP and [Met(O)¹⁵]-hBNP were delivered as a constant infusion via a syringe pump (Harvard Instrument, South Natick, Mass.) at escalating doses of 0.05, 0.1, and 0.2 μg/kg/min, for a period of 1 hour per dose. Blood samples were drawn 20 minutes prior to and 0, 50, 55, 60, 110, 115, 120, 170, 175, and 180 minutes following initiation of the infusion. For the intravenous bolus study, either hBNP, [Met(O)¹⁵]-hBNP, or [Met(O)⁴]-hBNP was administered as a single intravenous dose of 30 μg/kg (n=4 animals per compound). Blood samples were taken at 20 minutes prior to dosing, immediately prior to dosing and 5, 10, 15, 30, 60, and 90 minutes after dosing. Blood samples (3 mL) was drawn at each time point and the volume replaced with an equal volume of 0.9% NaCl. Blood was collected into EDTA coated tubes containing aprotinin (150 kallikrein inhibitory units/tube) and centrifuged immediately. The resulting plasma samples were stored at −80° C. until cyclic GMP and hBNP, [Met(O)¹⁵]-hBNP, and [Met(O)⁴]-hBNP determinations were made.

The steady state concentration for hBNP and [Met(O)¹⁵]-hBNP resulting from a dose (0.05, 0.1, and 0.2 μg/kg/min) escalating continuous intravenous infusion were 1319±615, 3434±952, and 7357±1569 pg/mL, respectively for hBNP, and 2119±882, 6578±1544, and 13344±3639 pg/mL, respectively for [Met(O)¹⁵]-hBNP. The pharmacokinetics of [Met(O)¹⁵]-hBNP derived from an intravenous bolus dose (30 μg/kg) also suggested increased plasma stability for this hBNP variant compared to hBNP. The pharmacokinetics of [Met(O)⁴]-hBNP derived from bolus administration was similar to what was measured for hBNP.

Plasma cyclic GMP levels were determined by radioimmunoassay (DuPont, Boston, Mass.). The labeled antigen was a succinyl tyrosine-(¹²⁵I)-methyl ester derivative of cyclic GMP. Separation of the bound from free antigen was performed using a pre-reacted primary and secondary antibody complex. Prior to the assay, the plasma samples were extracted with ethanol and the supernate evaporated to dryness in a speed vac concentrator (Savant Instruments, Holbrook, N.Y.). The dried samples were reconstituted with sodium acetate buffer. Plasma cyclic GMP levels were determined by interpolation from the standard curve (0.01 to 10 pmol/mL). Interassay and intraassay coefficients of variation were 0.18 and 0.27%, respectively.

Plasma hBNP, [Met(O)¹⁵]-hBNP, and [Met(O)⁴]-hBNP levels were determined by an antigen displacement enzyme immuno assay (EIA). The plasma samples were diluted appropriately with pooled normal rabbit plasma prior to assay. Monoclonal antibodies to hBNP were added to plasma samples and incubated overnight in microtitration plate wells precoated with Fc specific anti-murine antibodies. The amount of BNP, [Met(O)¹⁵]-hBNP, or [Met(O)⁴]-hBNP bound relative to the total binding capacity of the monoclonal antibodies was determined by assessing the binding of a biotinylated BNP probe. The concentration of hBNP, [Met(O)¹⁵]-hBNP, or [Met(O)⁴]-hBNP in the plasma samples was determined by comparing the amount of the biotin-BNP probe bound in the sample to the amount of probe in a reference matrix containing known amounts of hBNP, [Met(O)¹⁵]-hBNP, or [Met(O)⁴]-hBNP. Levels of the biotinylated probe were measured by using a sensitive enzyme based detection system, avidin-HRP, and a TMP substrate that provided a colorimetric endpoint.

[Met(O)¹⁵]-hBNP, administered either as a dose (0.05, 0.1, and 0.2 μg/kg/min) escalating continuous intravenous infusion or intravenous bolus (3 μg/kg), had significantly less activity than hBNP with regard to induction of plasma cyclic GMP in vivo. [Met(O)⁴]-hBNP, administered as a 30-μg/kg intravenous bolus, appeared to be equivalent to hBNP with regard to induction of plasma cyclic GMP.

[Met(O)¹⁵]-hBNP, administered to rabbits at 30 μg/kg, had significantly reduced renal effects when compared to hBNP, yet had similar hemodynamic effects.

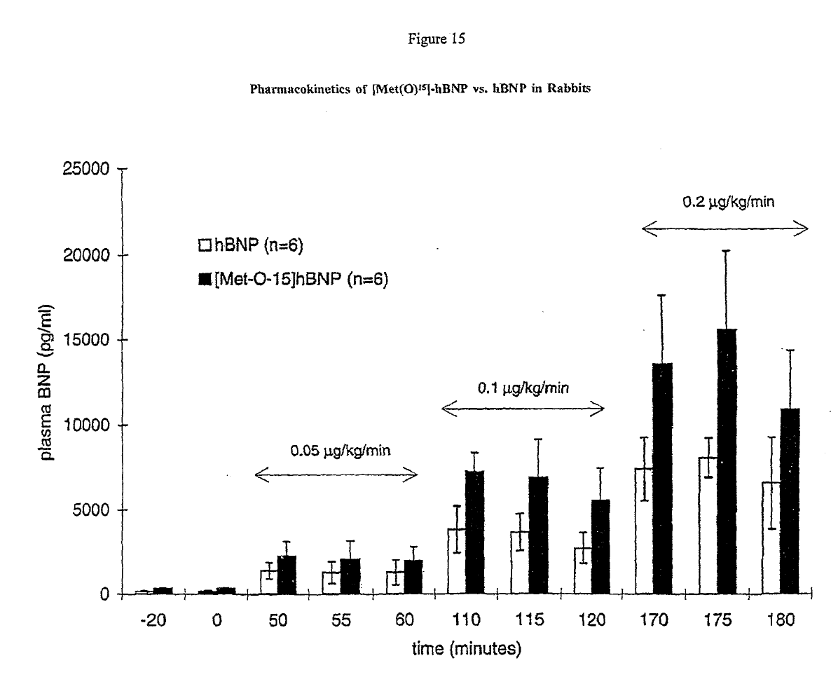

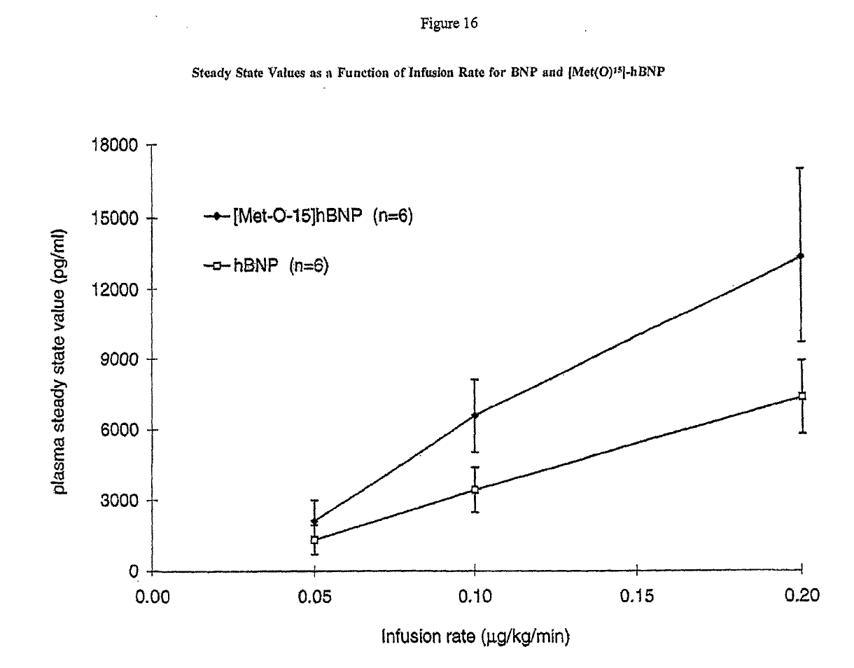

There was a dose-related increase in the plasma levels of [Met(O)¹⁵]-hBNP in rabbits during continuous intravenous infusion of 0.05, 0.1, and 0.2 μg/kg/min in rabbits (see FIG. 15 and Table 4). Plasma hBNP levels resulting from hBNP infusion at these same doses also increased in a dose-related manner (see FIG. 15). Plasma concentrations of [Met(O)¹⁵]-hBNP were greater than hBNP at infusion rates of 0.1 and 0.2 μg/kg/min (P<0.05) but not 0.05 μg/kg/min. The estimated plasma steady state concentrations for both [Met(O)¹⁵]-hBNP and hBNP were linearly related to dose (see FIG. 16). At each infusion rate, the estimated steady state value of [Met(O)¹⁵]-hBNP was greater than that of hBNP (see Table 5). This difference was statistically significant (P<0.005) at the two highest doses and not at the lowest dose.

Intravenous bolus administration of [Met(O)¹⁵]-hBNP, [Met(O)⁴]-hBNP, or hBNP resulted in a time dependent reduction in plasma levels (Table 6). At 15, 30, 60, and 90 minutes plasma concentrations of [Met(O)¹⁵]-hBNP were greater than those for hBNP(P<0.05). Plasma levels of [Met(O)⁴]-hBNP were less than that of hBNP at 5 minutes following bolus administration (P<0.05). At all other time points there were no remarkable differences between the plasma concentrations of [Met(O)⁴]-hBNP and hBNP.

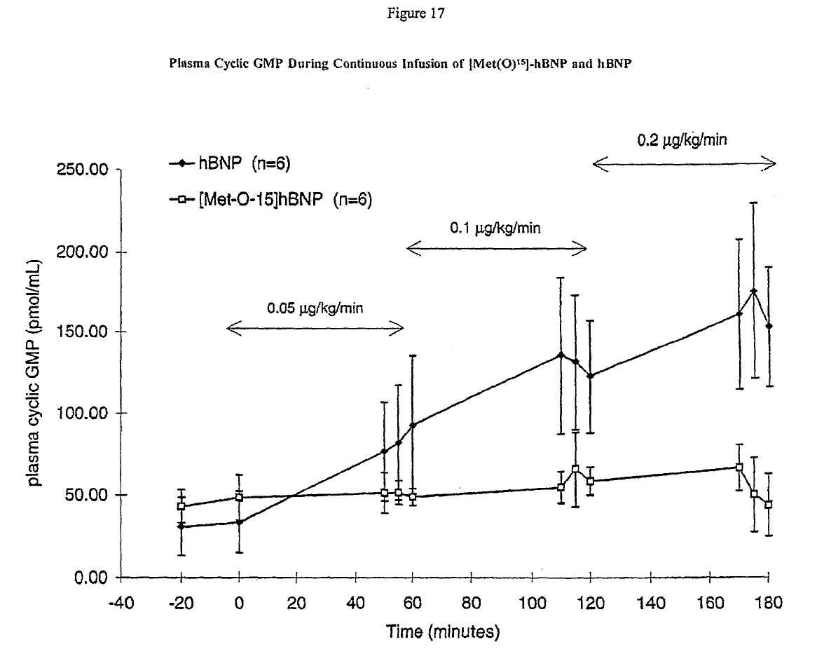

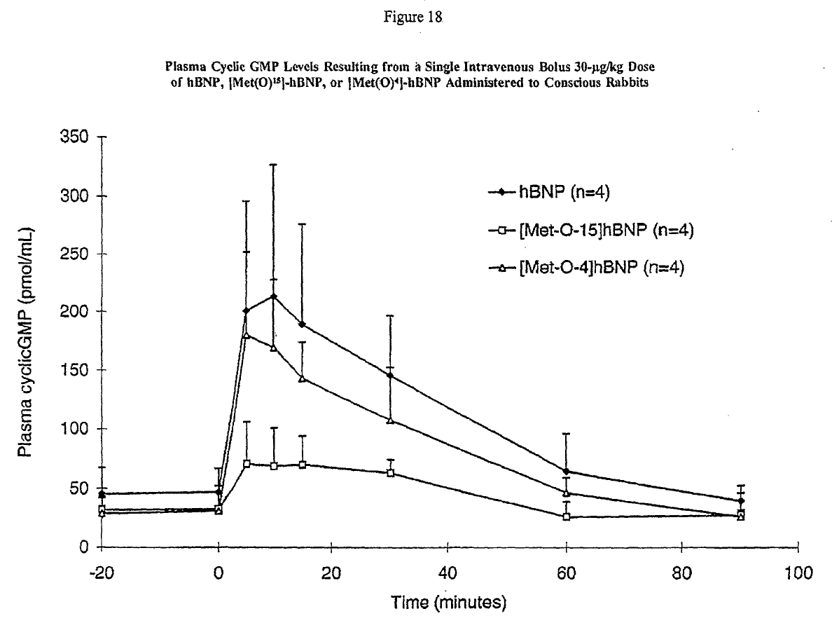

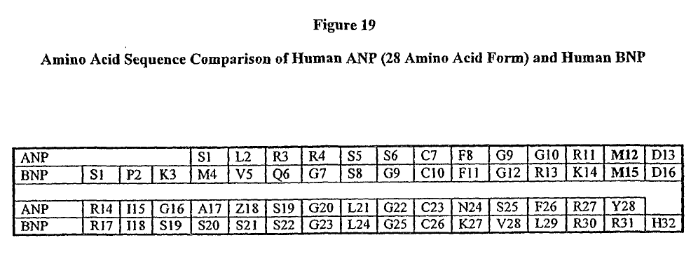

Animals treated with hBNP administered by continuous infusion had a dose-related increase in plasma cyclic GMP (FIG. 17 and Table 7). In contrast, there was no increase in plasma cyclic GMP in animals treated with [Met(O)¹⁵]-hBNP (FIG. 17 and Table 6). Following bolus administration of [Met(O)¹⁵]-hBNP, [Met(O)⁴]-hBNP, or hBNP there was a time dependent increase in plasma cyclic GMP (P<0.05) (FIG. 18 and Table 8). The levels of plasma cyclic GMP following [Met(O)⁴]-hBNP, and hBNP were comparable. The levels following [Met(O)¹⁵]-hBNP treatment were significantly less than what was measured following [Met(O)⁴]-hBNP, or hBNP treatment.

TABLE 4
MEAN PLASMA HBNP OR [MET(O)¹⁵]-HBNP LEVELS
RESULTING FROM CONTINUOUS INTRAVENOUS INFUSION
OF HBNP OR [MET(O)¹⁵]-HBNP ADMINISTERED IN A
DOSE-ESCALATING MANNER TO CONSCIOUS RABBITS

| | Time | hBNP | | [Met(O)¹⁵]-hBNP | |
|---|---|---|---|---|---|
| Infusion Rate | (min) | (pg/mL) | SD | pg/mL | SD |
| 0 μg/kg/min | −20 | 166 | 23 | 334 | 23 |
| | 0 | 146 | 63 | 335 | 26 |
| 0.05 μg/kg/min | 50 | 1390 | 483 | 2279 | 862 |
| | 55 | 1277 | 663 | 2091 | 1100 |
| | 60 | 1290 | 747 | 1987 | 865 |
| 0.1 μg/kg/min | 110 | 3855 | 1385 | 7238** | 1150 |
| | 115 | 3696 | 1096 | 6916* | 2255 |
| | 120 | 2751 | 926 | 5579* | 1879 |
| 0.2 μg/kg/min | 170 | 7402 | 1865 | 13544* | 4080 |
| | 175 | 8074 | 1168 | 15609** | 4631 |
| | 180 | 6595 | 2689 | 10880* | 3485 |

N = 6 per group
*P < 0.05
**P < 0.005 comparing value of plasma [Met(O)¹⁵]-hBNP to the value of plasma hBNP derived at the same point by unpaired, two tailed t test

TABLE 5
HBNP AND [MET(O)¹⁵]-HBNP: PLASMA STEADY STATE
CONCENTRATIONS AS A FUNCTION OF INFUSION RATE

| | [Met(O)¹⁵]-hBNP | hBNP |
|---|---|---|
| Infusion Rate | (n = 6) | (n = 6) |
| 0.05 μg/kg/min | 2119 ± 882 | 1319 ± 615 |
| 0.1 μg/kg/min | 6578 ± 1544** | 3434 ± 952 |
| 0.2 μg/kg/min | 13344 ± 3639** | 7357 ± 1569 |

TABLE 6
MEAN PLASMA HBNP, [MET(O)¹⁵]-HBNP, AND [MET(O)⁴]-HBNP
LEVELS RESULTING FROM A SINGLE INTRAVENOUS BOLUS 30
μG/KG DOSE OF HBNP [MET(O)¹⁵]-HBNP, OR [MET(O)⁴]-HBNP
ADMINISTERED TO CONSCIOUS RABBITS

| Time | hBNP | | [Met(O)¹⁵]- | | [Met(O)⁴]- | |
|---|---|---|---|---|---|---|
| (min) | (pg/mL) | SD | hBNP (pg/mL) | SD | hBNP (pg/mL) | SD |
| 5 | 50149 | 1662 | 52376 | 7364 | 29933* | 23522 |
| 10 | 12033 | 5112 | 12512 | 5412 | 17693 | 6323 |
| 15 | 4671 | 1734 | 8228 | 3543 | 10048 | 3706 |
| 30 | 1206 | 424 | 3373* | 591 | 1929 | 1254 |
| 60 | 122 | 20 | 194 | 59 | 89 | 73 |
| 90 | 51 | 18 | 110* | 25 | 61 | 38 |

n = 4 animals per group
*P < 0.05 vs hBNP values using one way analysis of variance with Bonferroni post test.

TABLE 7
PLASMA CYCLIC GMP LEVELS RESULTING FROM
CONTINUOUS INTRAVENOUS INFUSION OF HBNP OR
[MET(O)¹⁵]-HBNP ADMINISTERED IN A
DOSE-ESCALATING MANNER TO CONSCIOUS RABBITS

| | | Mean Plasma Cyclic GMP ± SD | |
|---|---|---|---|
| | | (pmol/mL) | |
| | Time | hBNP | [Met(O)¹⁵]-hBNP |
| Infusion Rate | (minutes) | (n = 6) | (n = 6) |
| 0 μg/kg/min (saline) | −20 | 34 ± 15 | 52 ± 23 |
| | 0 | 30 ± 16 | 58 ± 26 |
| 0.05 μg/kg/min | 50 | 68 ± 33 | 51 ± 16 |
| | 55 | 87 ± 29 | 54 ± 19 |
| | 60 | 92 ± 34 | 52 ± 16 |
| 0.1 μg/kg/min | 110 | 131 ± 38 | 53 ± 11 |
| | 115 | 122 ± 35 | 59 ± 21 |
| | 120 | 125 ± 27 | 58 ± 13 |
| 0.2 μg/kg/min | 170 | 159 ± 37 | 68 ± 11 |
| | 175 | 170 ± 43 | 61 ± 24 |
| | 180 | 145 ± 31 | 59 ± 28 |

n = 6 animals per group

TABLE 8
MEAN PLASMA CYCLIC GMP LEVELS RESULTING FROM
A SINGLE INTRAVENOUS BOLUS 30-μG/KG DOSE
OF HBNP, [MET(O)¹⁵]-HBNP, OR [MET(O)⁴]-HBNP
ADMINISTERED TO CONSCIOUS RABBITS

| Time | Plasma Cyclic GMP (pmol/mL) | | | | | |
|---|---|---|---|---|---|---|
| (min) | hBNP | SD | [Met(O)¹⁵]-hBNP | SD | [Met(O)⁴]-hBNP | SD |
| −20 | 46 | 22 | 32 | 11 | 29 | 13 |
| 0 | 47 | 20 | 33 | 20 | 31 | 16 |
| 5 | 201* | 95 | 71* | 35 | 180* | 72 |
| 10 | 214* | 113 | 69 | 32 | 169* | 59 |
| 15 | 189* | 88 | 70 | 24 | 144* | 30 |
| 30 | 146 | 51 | 63 | 11 | 108* | 45 |
| 60 | 65 | 31 | 26 | 13 | 47 | 13 |
| 90 | 40 | 13 | 28 | 19 | 26 | 6 |

n = 4 animals per group
*P < 0.05 comparing the plasma cyclic GMP value obtained at each time point to the value obtained at time 0 using repeated measures analysis of variance using Dunnett multiple comparisons test.

The hBNP variants [Met(O)⁴]-hBNP and [Met(O)¹⁵]-hBNP are present in the hBNP drug product. These impurities might by pharmacologically active as they are structurally similar to hBNP. This report describes the in vitro and in vivo pharmacology of [Met(O)⁴]-hBNP and [Met(O)¹⁵]-hBNP. In vitro, using cells expressing the human GC-A receptor, [Met(O)⁴]-hBNP was shown to be equivalent to hBNP in inducing cellular cyclic GMP release, a measure of receptor activation. [Met(O)¹⁵]-hBNP was far less potent than hBNP in this assay, indicating that it is a much poorer ligand for hBNP's biological receptor. These results are consistent with the animal data showing that with regard to induction of plasma cyclic GMP, a measure of GC-A receptor activation in vivo, [Met(O)⁴]-hBNP and hBNP were comparable while [Met(O)¹⁵]-hBNP was significantly less effective.

It appears that pharmacokinetics does not explain the weak induction of plasma cyclic GMP by [Met(O)¹⁵]-hBNP. In rabbits, [Met(O)¹⁵]-hBNP appeared to be more stable in the plasma compartment than hBNP as evidenced by the higher steady state plasma levels achieved at equivalent doses of these two peptides. The increased values for the estimated steady state of [Met(O)¹⁵]-hBNP compared to hBNP in rabbits suggest that [Met(O)¹⁵]-hBNP may achieve a higher plasma level in humans as well. As the estimated steady state values for [Met(O)¹⁵]-hBNP were between 1.6- and 1.9-fold greater than the values derived for hBNP using identical infusion rates, the predicted change in clearance rate in humans compared to hBNP is relatively small. There were no remarkable differences between the pharmacokinetics of [Met(O)⁴]-hBNP and hBNP.

While [Met(O)¹⁵]-hBNP had diuretic and natriuretic properties in rabbits, the magnitude of these effects tended to be lower than the effects of hBNP. There were no remarkable differences in the hemodynamic effects of [Met(O)¹⁵]-hBNP and hBNP, including reductions in systolic, diastolic, mean, and pulse pressures. The mechanisms by which [Met(O)¹⁵]-hBNP exerts diuretic, natriuretic and hemodynamic effects is not clear. It is possible that [Met(O)¹⁵]-hBNP binds to the natriuretic peptide clearance receptor, displacing endogenous natriuretic peptides that could exert these pharmacological effects. The natriuretic peptide clearance receptor has been shown to be much less restrictive in binding requirements for ligands as evidenced by the fact that is binds all three natriuretic peptides ANP, BNP and CNP as well as a wide variety of ANP analogs that do not activate the GC-A receptor. It is notable that, as described below, [Met-O¹²]ANP which like [Met(O)¹⁵]-hBNP has reduced activity for the GC-A receptor yet retains hemodynamic activity. It is also possible that the [Met(O)¹⁵]-hBNP peptide is 89% pure, and an impurity in this preparation could contribute to the hemodynamic effects noted here.

Previous in vitro and in vivo assays have shown that a single oxidation of the methionine residue at position 12 in human ANP, termed [Met-O12]ANP, significantly reduces the activity of the peptide. ANP and BNP are structurally similar, with the methionine at position 15 in human BNP corresponding to the methionine at position 12 in human ANP (FIG. 11). This region of the ANP peptide is believed to be important for activation of the (GC-A) receptor, the biological receptor for both ANP and BNP. Numerous reports have shown that [Met-O¹²]ANP has a reduced ability to stimulate cyclic GMP production in cultured cells expressing rat, murine, or human GC-A receptors. Using cells expressing the human GC-A receptor, researchers reported that [Met-O¹²]ANP had only 10% the potency of human ANP in stimulating cyclic GMP production (Mol Pharmacol, 1995; 47:172-80).

Consistent with these cell culture studies are reports that [Met-O¹²]ANP has reduced activity in relaxing pre-contracted vascular tissue preparations when compared to human ANP. In studies in intact rats [Met-O¹²]ANP has significantly reduced diuretic and natriuretic actions. It is interesting to note that in the one study in which blood pressure was measured, [Met-O¹²]ANP and human ANP had similar hypotensive actions. The low potency of [Met(O)¹⁵]-hBNP with regard to cyclic GMP induction and renal effects is consistent with what has been reported for [Met-O¹²]ANP. The reason for the normal hemodynamic effects of [Met-O¹²]ANP and [Met(O)¹⁵]-hBNP is unclear.

In summary, [Met(O)¹⁵]-hBNP has significantly reduced actions against hBNP's biological receptor. When compared to hBNP, [Met(O)¹⁵]-hBNP has reduced actions in rabbits with regard to induction of plasma cyclic GMP, natriuresis, and diuresis. However, it has hypotensive effects that appear to be similar to hBNP. [Met(O)⁴]-hBNP appears to be very similar to hBNP with regard to activity in vitro and in vivo.

What is claimed is:

1. An isolated and purified peptide having natriuretic activity, comprising: the formula:
Ser¹-Pro²-Lys³-Met⁴-Val⁵-Gln⁶-Gly⁷-Ser⁸-Gly⁹-Cys¹⁰-Phe¹¹-Gly¹²-Arg¹³-Lys¹⁴-Met¹⁵-Asp¹⁶-Arg¹⁷-Ile¹⁸-Ser¹⁹-Ser²⁰-Ser²¹-Ser²²-Gly²³-Leu²⁴-Gly²⁵-Cys²⁶-Lys²⁷-Val²⁸-Leu²⁹-Arg³⁰-Lys³¹-His³²
wherein either Met⁴, Met¹⁵, or both are oxidized.

2. A pharmaceutical composition for inducing natriuresis, diuresis and/or vasodilation in a subject which composition comprises an effective amount of the peptide of claim 1 in admixture with a suitable pharmaceutical excipient.

3. An isolated antibody or fragment thereof that is monospecifically reactive to hBNP, wherein the antibody binds specifically to a peptide of the formula:
Ser¹-Pro²-Lys³-Met⁴-Val⁵-Gln⁶-Gly⁷-Ser⁸-Gly⁹-Cys¹⁰-Phe¹¹-Gly¹²-Arg¹³-Lys¹⁴-Met¹⁵-Asp¹⁶-Arg¹⁷-Ile¹⁸-Ser¹⁹-Ser²⁰-Ser²¹-Ser²²-Gly²³-Leu²⁴-Gly²⁵Cys²⁶-Lys²⁷-Val²⁸-Leu²⁹-Arg³⁰-Lys³¹-His³²
wherein either Met⁴, Met¹⁵, or both are oxidized.

4. The antibody or fragment thereof of claims 3 or 4, wherein the antibody is monospecifically reactive with Met¹⁵-Asp¹⁶-Arg¹⁷-Ile¹⁸-Ser¹⁹-Ser²⁰-Ser²¹-Ser¹²-Gly²³-Leu²⁴-Gly²⁵.

5. The antibody or fragment thereof of claims 3 or 4, wherein the antibody is monospecifically reactive with Val⁵-Gln⁶-Gly⁷-Ser⁸-Gly⁹-Cys¹⁰-Phe¹¹-Gly¹²-Arg¹³.

6. The antibody or fragment thereof of claims 3, 4, or 5, wherein the antibody is a polyclonal antibody

7. The antibody or fragment thereof of claims 3, 4, or 5, wherein the antibody is a monoclonal antibody.

8. The antibody or fragment thereof of anyone of claims 3-6, wherein the fragment thereof is Fab, F(ab′)₂ and Fab′, Fv or sFv fragment

9. A method to diagnosis heart failure, comprising:
detecting the presence or absence of an oxidized form of hBNP in a sample, wherein the presence of oxidized hBNP is an indicator of heart failure.

10. The method of claim 8, wherein the oxidized form of hBNP is [Met(O)⁴]-hBNP.

11. The method of claim 8, wherein the oxidized form of hBNP is [Met(O)¹⁵]-hBNP.

12. The method of claim 8, wherein the oxidized form of hBNP is [Met(O)⁴, Met(O)¹⁵]-hBNP.

13. The method of claims 9-12, wherein the oxidized form of hBNP is detected using an immunoassay.

14. The method of claim 12, wherein the immunoassay employs an antibody according to anyone of claims 3-8.

15. A method to produce an oxidized hBNP peptide having natriuretic activity, comprising:
providing a recombinant host cell which has been manipulated to contain an expression system of which expresses the peptide of claim 1;
culturing the cells under conditions which permit the expression of the peptide;
recovering the peptide from the culture; and
oxidizing the peptide, such that Met⁴, Met¹⁵ or both are oxidized.

16. A method to treat a subject for a condition characterized by an abnormally high extracellular fluid level which method comprises:
administering to said subject an effective amount of the composition of claim 2, whereby extracellular fluid levels decrease.

17. A method of producing elevated levels of oxidize hBNP in plasma, comprising:
administering to a subject an effective amount of the composition of claim 2, whereby elevated plasma levels of the oxidized hBNP results.


SEQUENCE LISTING (5 sequences)

SEQ ID NO: 1 - protein, 134 aa, Homo sapiens
MDPQTAPSRALLLLLFLHLAFLGGRSHPLGSPGSASDLETSGLQEQRNHLQGKLSELQVEQTSLEPLQESPRPTGVWKSREVATEGIRGHRKMVLYTLRAPRSPKMVQGSGCFGRKMDRISSSSGLGCKVLRRH

SEQ ID NO: 2 - protein, 108 aa, Homo sapiens
HPLGSPGSASDLETSGLQEQRNHLQGKLSELQVEQTSLEPLQESPRPTGVWKSREVATEGIRGHRKMVLYTLRAPRSPKMVQGSGCFGRKMDRISSSSGLGCKVLRRH

SEQ ID NO: 3 - protein, 32 aa, Homo sapiens
SPKMVQGSGCFGRKMDRISSSSGLGCKVLRRH

SEQ ID NO: 4 - protein, 26 aa, Homo sapiens
GSGCFGRKMDRISSSSGLGCKVLRRH

SEQ ID NO: 5 - protein, 17 aa, Homo sapiens
CFGRKMDRISSSSGLGC